SEQUENCE LISTING (1 sequence)

SEQ ID NO: 1 - protein, 11 aa, synthetic construct
  REGION 1..11: note = source = /note= "Description of Artificial Sequence: Synthetic Peptide"
EDYHSLYQSHL

(19) United States
(12) Patent Application Publication
(10) Pub. No.: US 2025/0127700 A1
(43) Pub. Date: Apr. 24, 2025

(54) METHODS OF TREATING HYPERPIGMENTATION DISORDERS

(71) Applicant: DermBiont, Inc., Boston, MA (US)

(72) Inventors: Barbara GILCHREST, Boston, MA (US); Mark DE SOUZA, Berkeley, CA (US)

(21) Appl. No.: 18/897,336

(22) Filed: Sep. 26, 2024

Related U.S. Application Data
(63) Continuation of application No. 16/534,728, filed on Aug. 7, 2019, now abandoned.
Application No. 16/534,728 is a continuation of application No. 15/846,629, filed on Dec. 19, 2017, now abandoned.
(60) Provisional application No. 62/435,970, filed on Dec. 19, 2016.

(51) Int. Cl. A61K 8/49; A61K 9/00; A61K 31/407; A61Q 19/02

(52) U.S. Cl. CPC A61K 8/494; A61K 9/0014; A61K 31/407; A61Q 19/02

(57) ABSTRACT

Disclosed herein are compositions and methods of treating one or more symptom(s) related to a hyperpigmentation disorder.

### CROSS-REFERENCE

This application is a continuation of U.S. application Ser. No. 16/534,728, filed Aug. 7, 2019, which is a continuation of U.S. application Ser. No. 15/846,629, filed Dec. 19, 2017, which claims priority to U.S. Ser. No. 62/435,970 filed Dec. 19, 2016, the contents of each of which are incorporated herein by reference in their entireties.

### SEQUENCE LISTING

The instant application contains a Sequence Listing which has been submitted electronically and is hereby incorporated by reference in its entirety. Said copy, created on Sep. 26, 2024, is named 62826-713.302_SL and is 4,096 bytes in size.

### BACKGROUND

Melanin is the complex biopolymer that gives the skin and hair their natural pigment; generally the more melanin the darker the skin and hair. Melanin is produced by melanocytes and is derived from the amino acid tyrosine. The first and rate limiting step in the biosynthesis of melanin is the catalysis of L-3,4-dihydroxyphenylalanine by the oxidase tyrosinase, which is activated through phosphorylation by protein kinase C isoform β (PKCβ). Tyrosinase is expressed only in melanocytes. Skin pigmentation varies depending, inter alia, on the number and distribution of melanin containing melanosomes, and reflects the activity of tyrosinase in recent weeks. Without wishing to be bound by theory, it is believed that, when tyrosinase is mutated, e.g., non-functional, as in the disorder albinism, melanin production is reduced, e.g., minimal or no melanin is made.

Skin hyperpigmentation conditions are characterized by general or localized increases in an individual's normal skin color. Skin hyperpigmentation conditions include for example, melasma, post-inflammatory hyperpigmentation, discoid lupus erythematous and other forms of increased epidermal melanization. Melasma, for example, is characterized by dark, irregular well-demarcated hyperpigmented macules and patches commonly found on the upper cheek, upper nose, lip, and forehead, which often develop gradually over time. The hyperpigmentation can also appear on other parts of the body, particularly those subject to sunlight, such as the forearms and neck. Current therapies for melasma are all non-selective toxins that reduce tyrosinase activity, including hydroquinone (HQ), azaleic acid, and kojic acid. These current therapies are only partially effective and/or toxic; sometimes causing permanent pigment loss due to killing of melanocytes or ochronosis (a permanent dermal pigmentation condition characterized by erythema, bluish to black skin pigmentation, and papulo-nodular lesions). Thus, there is a need for novel methods of treating hyperpigmentation disorders, such as melasma, which are more effective, targeted, and which limit these serious side effects.

### SUMMARY OF THE INVENTION

The disclosure relates, inter alia, to novel methods of treating hyperpigmentation conditions or generally for decreasing unwanted pigmentation. For example, the disclosure provides novel methods of treating a hyperpigmentation condition, comprising, inter alia, non-systemically (e.g. topically, intradermally, or subcutaneously) administering an effective amount of a compound that selectively inhibits PKCβ, to the subject. The disclosure also relates, inter alia, to novel methods of decreasing pigmentation of the skin. For example, the disclosure provides novel methods of decreasing pigmentation of the skin comprising, non-systemically (e.g. topically, intradermally, or subcutaneously) administering an effective amount of a compound that selectively inhibits PKCβ, to the skin of a subject (e.g., a predetermined area of the skin of the subject).

In one aspect, the disclosure provides methods of decreasing skin pigmentation in a subject comprising, non-systemically (e.g. topically, intradermally, or subcutaneously) administering an effective amount of a compound that selectively inhibits PKCβ, to the subject, thereby decreasing skin pigmentation in the subject.

In some embodiments, decreasing skin pigmentation comprises decreasing skin pigmentation associated with a skin hyperpigmentation condition. In some embodiments, decreasing skin pigmentation comprises decreasing healthy or non-disease state skin pigmentation, e.g., unwanted healthy skin pigmentation.

In some embodiments, the compound comprises a compound of Formula I (e.g., as described herein). In some embodiments, the compound comprises a compound of Formula Id (e.g., as described herein). In some embodiments, the compound comprises a compound of Formula If-1 (e.g., as described herein). In some embodiments, the compound comprises a compound of Formula If-2 (e.g., as described herein). In some embodiments, the compound comprises ruboxistaurin or a salt thereof, e.g., ruboxistaurin mesylate. In some embodiments, the compound comprises ruboxistaurin mesylate. In some embodiments, the compound comprises ruboxistaurin formulated as a gel. In some embodiments, the compound comprises at least 0.05%, 0.1%, 0.2%, 0.3%, 0.4%, 0.5%, 0.6%, 0.7%, 0.8%, 0.9%, 1.0% or 1.5% ruboxistaurin, e.g., formulated as a gel. In some embodiments, the compound is administered topically. In some embodiments, the compound is administered intradermally. In some embodiments, the compound is administered subcutaneously.

In some embodiments, the compound comprising:
a compound of Formula I (e.g., as described herein);
a compound of Formula Id (e.g., as described herein);
a compound of Formula If-1 (e.g., as described herein);
a compound of Formula If-2 (e.g., as described herein);
ruboxistaurin or a salt thereof; or
ruboxistaurin mesylate,
reduces, e.g., inhibits, melanogenesis, e.g., skin darkening or pigmentation.

In some embodiments, melanogenesis is induced by, e.g., UV radiation exposure, e.g., sun exposure or a tanning response). In some embodiments, the compound reduces, e.g., inhibits, melanogenesis, e.g., skin darkening and pigmentation, by at least 10%, 20%, 30%, 40%, 50%, 60%, 70%, 80%, 90%, 95%, 99%, or 100%. In some embodiments, the reduction, e.g., inhibition, of melanogenesis is measured by an assay of Example 2.

In some embodiments, the compound comprises a compound of Formula I, Id, If-1, If-2, ruboxistaurin or a salt thereof, e.g., ruboxistaurin mesylate, e.g., formulated as a gel. In some embodiments, the formulation, e.g., gel formulation, comprises a dose of at least 0.05%, 0.1%, 0.2%, 0.3%, 0.4%, 0.5%, 0.6%, 0.7%, 0.8%, 0.9%, 1.0% or 1.5% ruboxistaurin or a salt thereof, e.g., ruboxistaurin mesylate. In some embodiments, the formulation, e.g., gel formulation, is suitable for topical administration.

In other embodiments, the compound comprises a bisindolylmaleimide or a derivative thereof. In embodiments, the bisindolylmaleimide derivative is chosen from: bisindolylmaleimide I (BIM-1), or bisindolylmaleimide II (BIM-2), or a salt thereof.

In some embodiments, administering comprises contacting a device comprising the compound, e.g., a dermal patch, with the subject, e.g., with the subject's skin. In some embodiments, administering comprises pretreating the skin to, e.g., enhance, penetration of the compound into skin, e.g., with a fraxel laser or micro-needle.

In some embodiments, the compound is administered in a pharmaceutical composition.

In some embodiments, the hyperpigmentation condition is chosen from: melasma, post inflammatory hyperpigmentation, discoid lupus erythematous, phytophotodermatitis, lentigines, (e.g., age spots), birth marks, café au lait macules, acanthosis nigricans, burn associated hyperpigmentation, drug-induced hyperpigmentation (e.g., sulfonamide, tetracycline, NSAID, barbiturate, and carbamazepine induced hyperpigmentation), injury induced hyperpigmentation, primary biliary cirrhosis associated hyperpigmentation, Addison's disease associated hyperpigmentation, melanocytic naevi, ephelides, seborrhoeic keratosis, skin cancer associated hyperpigmentation, infection associated hyperpigmentation (e.g., pityriasis versicolor, erythrasma), eczema, photocontact dermatitis, ichthyosis, neurofibromatosis, or hyperpigmentation associated with ultra-violet (UV) radiation exposure, e.g., sun exposure or a tanning response.

In some embodiments, the hyperpigmentation condition is melasma. In some embodiments, the hyperpigmentation condition is post inflammatory hyperpigmentation. In some embodiments, the hyperpigmentation condition is lentigines, e.g., age spots. In some embodiments, the hyperpigmentation condition is a tanning response.

In some embodiments, the compound is administered in combination with an additional agent, e.g., a therapeutic or cosmetic agent. In some embodiments, the one or more additional agent, e.g., therapeutic agents, is chosen from: hydroquinone, tretinoin, a corticosteroid, azaleic acid, kojic acid, a retinoid, glycolic acid, L-ascorbic acid, p-aminobenzoic acid, padimate O, phenylbenzimidazole sulfonic acid, cinoxate, menthyl anthranilate, dioxybenzone, oxybenzone, avobenzone, octisalate, octocrylene, octyl methoxycinnamate, homosalate, octinoxate, sulisobenzone, trolamine salicylate, ecamsule, zinc oxide, titanium dioxide, cosmetic agent, pigment, fragrance, sunscreen, a lathering surfactant, a vitamin, a hydroxy acid, an antioxidant, a retinoid, or a moisturizing agent.

In some embodiments, the one or more additional agent is administered topically. In some embodiments, the additional agent is co-applied with the compound. In some embodiments, the additional agent and the compound are applied to a common area but at different times. In some embodiments, the additional agent and the compound are comprised in the same unit dosage form. In some embodiments, the additional agent and the compound are disposed in different unit dosage forms. In some embodiments, a second or a plurality of additional agents are administered. In some embodiments, a second administration of the compound is administered. In some embodiments, a third administration of the compound is administered.

In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a seven day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a fourteen day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a twenty one day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a thirty day period.

In some embodiments, the compound is administered, daily, every other day, weekly, or monthly. In some embodiments, the compound is administered daily for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19months, 20 months, 21 months, 22 months, 23 months or longer. In some embodiments, the compound is administered daily for at least 1, 2, 3, 4, 5, 6, 7, 8, 9, or 10 years.

In some embodiments, the subject has previously been treated with the compound, e.g., had been treated for at least 1, 6, 12, 24, 36, or 48 months. In some embodiments, the subject has received one or more previous administrations of the compound, e.g., at least 2, 10, 20, 30, 40, 50, 100, 200, 300, or 500 previous administrations of the compound.

In some embodiments, the method comprises applying the compound to a predetermined region of the body, e.g., a predetermined region of skin, e.g., as described herein. In some embodiments, the method comprises applying the compound to an area of the skin affected by the hyperpigmentation condition. In some embodiments, the method comprises applying the compound to an area adjacent to an area of the skin affected by the hyperpigmentation condition. In some embodiments, the method comprises applying the compound to the face, arms, back, or other areas affected by unwanted or otherwise excess hyperpigmentation.

In one aspect, the disclosure provides methods of decreasing skin pigmentation associated with a skin hyperpigmentation condition of a subject, comprising, topically administering an effective amount of ruboxistaurin, to the area of the skin of the subject (e.g., a predetermined area of the skin of the subject), thereby decreasing the pigmentation of the skin the subject, relative to the pigmentation of the skin prior to the administration of the compound. In some embodiments, the hyperpigmentation condition comprises melasma.

In some embodiments, the compound comprises a compound of Formula I (e.g., as described herein). In some embodiments, the compound comprises a compound of Formula Id (e.g., as described herein). In some embodiments, the compound comprises a compound of Formula If-1 (e.g., as described herein). In some embodiments, the compound comprises a compound of Formula If-2 (e.g., as described herein). In some embodiments, the compound comprises ruboxistaurin or a salt thereof, e.g., ruboxistaurin mesylate. In some embodiments, the compound comprises ruboxistaurin mesylate. In some embodiments, the compound comprises ruboxistaurin formulated as a gel. In some embodiments, the compound comprises at least 0.05%, 0.1%, 0.2%, 0.3%, 0.4%, 0.5%, 0.6%, 0.7%, 0.8%, 0.9%, 1.0% or 1.5% ruboxistaurin, e.g., formulated as a gel. In some embodiments, the compound is administered topically. In some embodiments, the compound is administered intradermally. In some embodiments, the compound is administered subcutaneously.

In some embodiments, the compound comprising:
a compound of Formula I (e.g., as described herein);
a compound of Formula Id (e.g., as described herein);
a compound of Formula If-1 (e.g., as described herein);
a compound of Formula If-2 (e.g., as described herein);
ruboxistaurin or a salt thereof; or
ruboxistaurin mesylate,
reduces, e.g., inhibits, melanogenesis, e.g., skin darkening or pigmentation.

In some embodiments, melanogenesis is induced by, e.g., UV radiation exposure, e.g., sun exposure or a tanning response. In some embodiments, the compound reduces, e.g., inhibits, melanogenesis, e.g., skin darkening and pigmentation, by at least 10%, 20%, 30%, 40%, 50%, 60%, 70%, 80%, 90%, 95%, 99%, or 100%. In some embodiments, the reduction, e.g., inhibition, of melanogenesis is measured by an assay of Example 2.

In some embodiments, the compound comprises a compound of Formula I, Id, If-1, If-2, ruboxistaurin or a salt thereof, e.g., ruboxistaurin mesylate, e.g., formulated as a gel. In some embodiments, the formulation, e.g., gel formulation, comprises a dose of at least 0.05%, 0.1%, 0.2%, 0.3%, 0.4%, 0.5%, 0.6%, 0.7%, 0.8%, 0.9%, 1.0% or 1.5% ruboxistaurin or a salt thereof, e.g., ruboxistaurin mesylate. In some embodiments, the formulation, e.g., gel formulation, is suitable for topical administration.

In other embodiments, the compound comprises a bisindolylmaleimide or a derivative thereof. In embodiments, the bisindolylmaleimide derivative is chosen from: bisindolylmaleimide I (BIM-1), or bisindolylmaleimide II (BIM-2), or a salt thereof.

In some embodiments, the compound is administered in combination with an additional agent, e.g., a therapeutic or cosmetic agent. In some embodiments, the one or more additional agent, e.g., therapeutic agents, is chosen from: hydroquinone, tretinoin, a corticosteroid, azaleic acid, kojic acid, a retinoid, glycolic acid, L-ascorbic acid, p-aminobenzoic acid, padimate O, phenylbenzimidazole sulfonic acid, cinoxate, menthyl anthranilate, dioxybenzone, oxybenzone, avobenzone, octisalate, octocrylene, octyl methoxycinnamate, homosalate, octinoxate, sulisobenzone, trolamine salicylate, ecamsule, zinc oxide, titanium dioxide, cosmetic agent, pigment, fragrance, sunscreen, a lathering surfactant, a vitamin, a hydroxy acid, an antioxidant, a retinoid, or a moisturizing agent.

In some embodiments, the one or more additional agent is administered topically. In some embodiments, the additional agent is co-applied with the compound. In some embodiments, the additional agent and the compound are applied to a common area but at different times. In some embodiments, the additional agent and the compound are comprised in the same unit dosage form. In some embodiments, the additional agent and the compound are disposed in different unit dosage forms. In some embodiments, a second or a plurality of additional agents are administered. In some embodiments, a second administration of the compound is administered. In some embodiments, a third administration of the compound is administered.

In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a seven day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a fourteen day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a twenty one day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a thirty day period.

In some embodiments, the compound is administered, daily, every other day, weekly, or monthly. In some embodiments, the compound is administered daily for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months or longer. In some embodiments, the compound is administered daily for at least 1, 2, 3, 4, 5, 6, 7, 8, 9, or 10 years.

In some embodiments, the subject has previously been treated with the compound, e.g., had been treated for at least 1, 6, 12, 24, 36, or 48 months. In some embodiments, the subject has received one or more previous administrations of the compound, e.g., at least 2, 10, 20, 30, 40, 50, 100, 200, 300, or 500 previous administrations of the compound.

In some embodiments, the method comprises applying the compound to a predetermined region of the body, e.g., a predetermined area of skin. In some embodiments, the method comprises applying the compound to an area of the skin affected by the hyperpigmentation condition. In some embodiments, the method comprises applying the compound to an area adjacent to an area of the skin affected by the hyperpigmentation condition. In some embodiments, the method comprises applying the compound to the face, arms, back, or other areas affected by unwanted or otherwise excess hyperpigmentation.

In one aspect, the disclosure provides methods of decreasing healthy pigmentation of an area of skin of a subject, comprising, topically administering an effective amount of ruboxistaurin, to the area of the skin of the subject (e.g., a predetermined area of the skin of the subject), thereby decreasing the pigmentation of the skin the subject, relative to the pigmentation of the skin prior to the administration of the compound.

In some embodiments, the compound comprises a compound of Formula I (e.g., as described herein). In some embodiments, the compound comprises a compound of Formula Id (e.g., as described herein). In some embodiments, the compound comprises a compound of Formula If-1 (e.g., as described herein). In some embodiments, the compound comprises a compound of Formula If-2 (e.g., as described herein). In some embodiments, the compound comprises ruboxistaurin mesylate. In some embodiments, the compound comprises ruboxistaurin. In some embodiments, the compound comprises ruboxistaurin or a salt thereof, e.g., ruboxistaurin mesylate, e.g., formulated as a gel. In some embodiments, the compound comprises at least 0.05%, 0.1%, 0.2%, 0.3%, 0.4%, 0.5%, 0.6%, 0.7%, 0.8%, 0.9%, 1.0% or 1.5% ruboxistaurin or a salt thereof, e.g., ruboxistaurin mesylate, e.g., formulated as a gel. In some embodiments, the compound is administered topically. In some embodiments, the compound is administered intradermally. In some embodiments, the compound is administered subcutaneously.

In some embodiments, the compound comprising:
a compound of Formula I (e.g., as described herein);
a compound of Formula Id (e.g., as described herein);
a compound of Formula If-1 (e.g., as described herein);
a compound of Formula If-2 (e.g., as described herein);
ruboxistaurin or a salt thereof; or
ruboxistaurin mesylate,
reduces, e.g., inhibits, melanogenesis, e.g., skin darkening or pigmentation.

In some embodiments, melanogenesis is induced by, e.g., UV radiation exposure, e.g., sun exposure or a tanning response. In some embodiments, the compound reduces, e.g., inhibits, melanogenesis, e.g., skin darkening and pigmentation, by at least 10%, 20%, 30%, 40%, 50%, 60%, 70%, 80%, 90%, 95%, 99%, or 100%. In some embodiments, the reduction, e.g., inhibition, of melanogenesis is measured by an assay of Example 2.

In some embodiments, the compound comprises a compound of Formula I, Id, If-1, If-2, ruboxistaurin or a salt thereof, e.g., ruboxistaurin mesylate, e.g., formulated as a gel. In some embodiments, the formulation, e.g., gel formulation, comprises a dose of at least 0.05%, 0.1%, 0.2%, 0.3%, 0.4%, 0.5%, 0.6%, 0.7%, 0.8%, 0.9%, 1.0% or 1.5% ruboxistaurin or a salt thereof, e.g., ruboxistaurin mesylate. In some embodiments, the formulation, e.g., gel formulation, is suitable for topical administration.

In other embodiments, the compound comprises a bisindolylmaleimide or a derivative thereof. In embodiments, the bisindolylmaleimide derivative is chosen from: bisindolylmaleimide I (BIM-1), or bisindolylmaleimide II (BIM-2), or a salt thereof.

In some embodiments, the compound is administered in combination with an additional agent, e.g., a therapeutic or cosmetic agent. In some embodiments, the one or more additional agent, e.g., therapeutic agents, is chosen from: hydroquinone, tretinoin, a corticosteroid, azaleic acid, kojic acid, a retinoid, glycolic acid, L-ascorbic acid, p-aminobenzoic acid, padimate O, phenylbenzimidazole sulfonic acid, cinoxate, menthyl anthranilate, dioxybenzone, oxybenzone, avobenzone, octisalate, octocrylene, octyl methoxycinnamate, homosalate, octinoxate, sulisobenzone, trolamine salicylate, ecamsule, zinc oxide, titanium dioxide, cosmetic agent, pigment, fragrance, sunscreen, a lathering surfactant, a vitamin, a hydroxy acid, an antioxidant, a retinoid, or a moisturizing agent.

In some embodiments, the one or more additional agent is administered topically. In some embodiments, the additional agent is co-applied with the compound. In some embodiments, the additional agent and the compound are applied to a common area but at different times. In some embodiments, the additional agent and the compound are comprised in the same unit dosage form. In some embodiments, the additional agent and the compound are disposed in different unit dosage forms. In some embodiments, a second or a plurality of additional agents are administered. In some embodiments, a second administration of the compound is administered. In some embodiments, a third administration of the compound is administered.

In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a seven day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a fourteen day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a twenty one day period. In some embodiments, a plurality of, e.g.,2 or more, 3 or more, 4 or more, 5 or more, 6 or more, 7 or more, 8 or more, 9 or more, 10 or more, 11 or more, 12 or more, 13 or more, 14 or more, or 15 or more administrations in a thirty day period.

In some embodiments, the compound is administered, daily, every other day, weekly, or monthly. In some embodiments, the compound is administered daily for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months or longer. In some embodiments, the compound is administered daily for at least 1, 2, 3, 4, 5, 6, 7, 8, 9, or 10 years.

In some embodiments, the subject has previously been treated with the compound, e.g., had been treated for at least 1, 6, 12, 24, 36, or 48 months. In some embodiments, the subject has received one or more previous administrations of the compound, e.g., at least 2, 10, 20, 30, 40, 50, 100, 200, 300, or 500 previous administrations of the compound.

In some embodiments, the method comprises applying the compound to a predetermined region of the body, e.g., a predetermined region of skin, e.g., as described herein. In some embodiments, the method comprises applying the compound to an area of the skin affected by the hyperpigmentation condition. In some embodiments, the method comprises applying the compound to an area adjacent to an area of the skin affected by the hyperpigmentation condition. In some embodiments, the method comprises applying the compound to the face, arms, back, or other areas affected by unwanted or otherwise excess hyperpigmentation.

In one aspect, the disclosure provides methods of decreasing pigmentation of a growing hair, of a subject, comprising, non-systemically (e.g. topically, intradermally, or subcutaneously) applying an effective amount of a compound that selectively inhibits PKCβ, to hair bearing skin of the subject (e.g., at follicular orifices surrounding emerging hair shafts), thereby decreasing the pigmentation of the growing hair (e.g., decreased relative to the pigmentation of non-growing hair of the subject).

In some embodiments, the subject has artificially colored, e.g., dyed, hair. In some embodiments, the compound is administered in a regimen that is effective to decrease the difference in color or shade of a newly grown portion of the hair shaft and an older portion of the hair shaft, e.g., in a subject with artificially colored hair. In some embodiments, administration of the compound increases the interval, e.g., time period, in which artificial color, e.g., dye, is applied, e.g., reapplied, to hair. In some embodiments, the interval is increased by at least 1 week, 2 weeks, 3 weeks, 4 weeks, 5 weeks, 6 weeks, 7 weeks or 8 weeks.

In some embodiments, the compound comprises a compound of Formula I (e.g., as described herein). In some embodiments, the compound comprises a compound of Formula Id (e.g., as described herein). In some embodiments, the compound comprises a compound of Formula If-1 (e.g., as described herein). In some embodiments, the compound comprises a compound of Formula If-2 (e.g., as described herein). In some embodiments, the compound comprises ruboxistaurin mesylate. In some embodiments, the compound comprises ruboxistaurin. In some embodiments, the compound comprises ruboxistaurin or a salt thereof, e.g., ruboxistaurin mesylate, e.g., formulated as a gel. In some embodiments, the compound comprises at least 0.05%, 0.1%, 0.2%, 0.3%, 0.4%, 0.5%, 0.6%, 0.7%, 0.8%, 0.9%, 1.0% or 1.5% ruboxistaurin or a salt thereof, e.g., ruboxistaurin mesylate, e.g., formulated as a gel. In some embodiments, the compound is administered topically. In some embodiments, the compound is administered intradermally. In some embodiments, the compound is administered subcutaneously.

In some embodiments, the compound comprising:
a compound of Formula I (e.g., as described herein);
a compound of Formula Id (e.g., as described herein);
a compound of Formula If-1 (e.g., as described herein);
a compound of Formula If-2 (e.g., as described herein);
ruboxistaurin or a salt thereof; or
ruboxistaurin mesylate,
reduces, e.g., inhibits, melanogenesis, e.g., skin darkening or pigmentation.

In some embodiments, melanogenesis is induced by, e.g., UV radiation exposure, e.g., sun exposure or a tanning response). In some embodiments, the compound reduces, e.g., inhibits, melanogenesis, e.g., skin darkening and pigmentation, by at least 10%, 20%, 30%, 40%, 50%, 60%, 70%, 80%, 90%, 95%, 99%, or 100%. In some embodiments, the reduction, e.g., inhibition, of melanogenesis is measured by an assay of Example 2.

In some embodiments, the compound comprises a compound of Formula I, Id, If-1, If-2, ruboxistaurin or a salt thereof, e.g., ruboxistaurin mesylate, e.g., formulated as a gel. In some embodiments, the formulation, e.g., gel formulation, comprises a dose of at least 0.05%, 0.1%, 0.2%, 0.3%, 0.4%, 0.5%, 0.6%, 0.7%, 0.8%, 0.9%, 1.0% or 1.5% ruboxistaurin or a salt thereof, e.g., ruboxistaurin mesylate. In some embodiments, the formulation, e.g., gel formulation, is suitable for topical administration.

In other embodiments, the compound comprises a bisindolylmaleimide or a derivative thereof. In embodiments, the bisindolylmaleimide derivative is chosen from: bisindolylmaleimide I (BIM-1), or bisindolylmaleimide II (BIM-2), or a salt thereof.

In some embodiments, the compound is administered in combination with an additional agent, e.g., a therapeutic or cosmetic agent. In some embodiments, the one or more additional agent, e.g., therapeutic agents, is chosen from: hydroquinone, tretinoin, a corticosteroid, azaleic acid, kojic acid, a retinoid, glycolic acid, L-ascorbic acid, p-aminobenzoic acid, padimate O, phenylbenzimidazole sulfonic acid, cinoxate, menthyl anthranilate, dioxybenzone, oxybenzone, avobenzone, octisalate, octocrylene, octyl methoxycinnamate, homosalate, octinoxate, sulisobenzone, trolamine salicylate, ecamsule, zinc oxide, titanium dioxide, cosmetic agent, pigment, fragrance, sunscreen, a lathering surfactant, a vitamin, a hydroxy acid, an antioxidant, a retinoid, or a moisturizing agent.

In some embodiments, the one or more additional agent is administered topically. In some embodiments, the additional agent is co-applied with the compound. In some embodiments, the additional agent and the compound are applied to a common area but at different times. In some embodiments, the additional agent and the compound are comprised in the same unit dosage form. In some embodiments, the additional agent and the compound are disposed in different unit dosage forms. In some embodiments, a second or a plurality of additional agents are administered. In some embodiments, a second administration of the compound is administered. In some embodiments, a third administration of the compound is administered.

In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a seven day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a fourteen day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a twenty one day period. In some embodiments, a plurality of, e.g., 2 or more, 3 or more, 4 or more, 5 or more, 6 or more, 7 or more, 8 or more, 9 or more, 10 or more, 11 or more, 12 or more, 13 or more, 14 or more, or 15 or more administrations in a thirty day period.

In some embodiments, the compound is administered, daily, every other day, weekly, or monthly. In some embodiments, the compound is administered daily for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months or longer. In some embodiments, the compound is administered daily for at least 1, 2, 3, 4, 5, 6, 7, 8, 9, or 10 years.

In some embodiments, the subject has previously been treated with the compound, e.g., had been treated for at least 1, 6, 12, 24, 36, or 48 months. In some embodiments, the subject has received one or more previous administrations of the compound, e.g., at least 2, 10, 20, 30, 40, 50, 100, 200, 300, or 500 previous administrations of the compound.

In some embodiments, the method comprises applying the compound to a predetermined region of the body, e.g., a predetermined region of skin, e.g., as described herein. In some embodiments, the method comprises applying the compound to an area of the skin affected by the hyperpigmentation condition. In some embodiments, the method comprises applying the compound to an area adjacent to an area of the skin affected by the hyperpigmentation condition. In some embodiments, the method comprises applying the compound to the face, arms, back, or other areas affected by unwanted or otherwise excess hyperpigmentation.

In an aspect, provided herein, is a unit dosage form or formulation comprising a compound comprising:
a compound of Formula I (e.g., as described herein);
a compound of Formula Id (e.g., as described herein);
a compound of Formula If-1 (e.g., as described herein);
a compound of Formula If-2 (e.g., as described herein);
ruboxistaurin or a salt thereof; or
ruboxistaurin mesylate,
wherein the compound in the unit dosage form or the formulation is in an amount sufficient to reduce, e.g., inhibit, melanogenesis, e.g., skin darkening or pigmentation. In embodiments, the compound in the unit dosage form or the formulation, e.g., a gel formulation, is present at a dose of at least 0.05%, 0.1%, 0.2%, 0.3%, 0.4%, 0.5%, 0.6%, 0.7%, 0.8%, 0.9%, 1.0% or 1.5% of any of the compounds of Formula I, Id, If-1, If-2, ruboxistaurin or a salt thereof, e.g., ruboxistaurin mesylate. In embodiments, the unit dosage form or the formulation, e.g., a gel formulation, comprises ruboxistaurin or a salt thereof, e.g., ruboxistaurin mesylate, at a dose of at least 0.1%, 0.2%, 0.3%, 0.4%, 0.5%, 0.6%, 0.7%, 0.8%, 0.9%, 1.0% or 1.5%. In embodiments, the unit dosage form or the formulation, e.g., gel formulation, of any of the compounds of Formula I, Id, If-1, If-2, ruboxistaurin or a salt thereof, e.g., ruboxistaurin mesylate, is suitable for topical administration.

In some embodiments, melanogenesis is induced by, e.g., UV radiation exposure, e.g., sun exposure or a tanning response. In some embodiments, the compound reduces, e.g., inhibits, melanogenesis, e.g., skin darkening and pigmentation, by at least 10%, 20%, 30%, 40%, 50%, 60%, 70%, 80%, 90%, 95%, 99%, or 100%. In some embodiments, the reduction, e.g., inhibition, of melanogenesis is measured by an assay of Example 2.

In one aspect, the disclosure provides a set of unit dosage forms comprising: a first unit dosage form comprising an effective amount of a compound that selectively inhibits PKCβ formulated for non-systemic administration; and a second unit dosage form comprising an effective amount of an additional agent, e.g., a therapeutic or cosmetic agent. In some embodiments, the additional agent comprises an agent chosen from hydroquinone, tretinoin, a corticosteroid, azaleic acid, kojic acid, a retinoid, glycolic acid, L-ascorbic acid, p-aminobenzoic acid, padimate O, phenylbenzimidazole sulfonic acid, cinoxate, menthyl anthranilate, dioxybenzone, oxybenzone, avobenzone, octisalate, octocrylene, octyl methoxycinnamate, homosalate, octinoxate, sulisobenzone, trolamine salicylate, ecamsule, zinc oxide, titanium dioxide, cosmetic agent, pigment, fragrance, sunscreen, a lathering surfactant, a vitamin, a hydroxy acid, an antioxidant, a retinoid, or a moisturizing agent.

In one aspect, the disclosure provides pharmaceutical compositions for use in the methods described herein. In some embodiments, the pharmaceutical composition is formulated as a lotion, cream, serum, spray, mousse, aerosol, emulsion, cake, ointment, gel, paste, patch, pencil, towelette, mask, stick, foam, elixir, or concentrate. In some embodiments, the pharmaceutical composition further comprises one or more additional agent, e.g., therapeutic agents, chosen from: hydroquinone, tretinoin, a corticosteroid, azaleic acid, kojic acid, a retinoid, glycolic acid, L-ascorbic acid, p-aminobenzoic acid, padimate O, phenylbenzimidazole sulfonic acid, cinoxate, menthyl anthranilate, dioxybenzone, oxybenzone, avobenzone, octisalate, octocrylene, octyl methoxycinnamate, homosalate, octinoxate, sulisobenzone, trolamine salicylate, ecamsule, zinc oxide, titanium dioxide, cosmetic agent, pigment, fragrance, sunscreen, a lathering surfactant, a vitamin, a hydroxy acid, an antioxidant, a retinoid, or a moisturizing agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a micrograph of awl and zig-zag hairs collected from dorsal mouse skin. 8-9 week old mice were wax depilated to completely remove all hairs and stimulate the follicles to being a new cycle of hair growth and treated with vehicle (DMSO:PG) alone (A) 4% HQ (B), bisindolylmaleimide (Bis), a non-selective PKC inhibitor (C), or ruboxistaurin, a highly selective PKCB inhibitor (D). 21 days after depilation all groups of treated hairs appeared lighter compared to the control group (A). Zig-zag hairs (the most numerous hair shaft type ad those with the more superficial follicles (residing in the mid-dermis) from both Bis and ruboxistaurin treated groups were lightest.

FIG. 2 is a bar graph depicting the effect of topically applied compounds on hair pigmentation density (reflecting the amount of melanin produced in follicular melanocytes and transferred to follicular keratinocytes that then differentiate to form the hair shaft). Average gray value within the hair shafts were determined using ImageJ (NIH) software. The gray value is the sum of the gray values of all the pixels in the hair shaft divided by the number of pixels; and values range from 0 (white, no pigment) to 255 (black). Small hair follicles (B) exhibit significantly higher lightening effects in all treated groups than large hairs (A). The median of the gray value of small hairs is indicated by red line. Students t-test (*<0.05; **<0.01).

FIG. 3 is a bar graph depicting the maximum lightening effect of topically applied compounds on small hairs. Dark and light hairs were selected for separate analysis based on the median gray value of the main group of small hairs. The hairs with a value above and below the median were considered as dark (A) and light (B), respectively. Students t-test (*<0.05; **<0.01; ***<0.001).

FIG. 4 is a graph depicting results of Investigator Dynamic Grading Assessment (IDGA) of test areas in subjects treated with ruboxistaurin gel or placebo. Three doses of ruboxistaurin gel formulated as a gel were administered: 0.1%, 0.5% or 1.0%. The y-axis depicts the IDGA score (as described in Table 2). Mean values of all test sites from all subjects were plotted, with error bars indicating standard deviation.

### DETAILED DESCRIPTION OF THE INVENTION

The protein kinase C (PKC) family of proteins is a group of serine/threonine kinases comprising at least 11 different isoforms, including e.g., PKCα, PKCβ (PKCβ1 and PKCβ2), PKCγ, PKCδ, PKCε, PKCζ, PKCι, PKCθ, and PKCη. Both PKCβ and PKCα are expressed in the skin; however PKCβ is expressed only in melanocytes; while PKCα is expressed in melanocytes, keratinocytes, and fibroblasts. Skin hyperpigmentation disorders are characterized by general or localized increases in melanin (increased relative to the individual's normal or average skin pigment), producing one or more areas of skin with increased pigment (increased relative to the individual's normal or average skin pigment). Tyrosinase mediates the first and rate limiting step in melanin biosynthesis, and is activated through phosphorylation by PKCβ.

The present disclosure provides for novel methods of treating hyperpigmentation conditions using local or non-systemic (e.g., topical, intradermal, subcutaneous) administration of a selective PKCβ inhibitor (e.g., a compound described herein, e.g., a compound of Formula I, a compound of Formula II, or ruboxistaurin). While bisindolylmaleimide (Bis), a non-selective PKC inhibitor, has been described in the art (e.g., Park et al. (2004) J of Investigative Dermatology 122:159-166; and U.S. Pat. No. 5,962,417), the compounds of the present disclosure are selective PKCβ inhibitors (e.g., a compound described herein, e.g., a compound of Formula I, a compound of Formula II, or ruboxistaurin). Bis inhibits the activity of several PKC isoforms, e.g., Bis has a PKCβ IC₅₀=18 nM; PKCα IC₅₀=8.4 nM; and PKCζ IC₅₀=5.8 μM) (Toullec, D., et al. 1991. J. Biol. Chem. 266:15771-15781. PMID: 1874734; Martiny-Baron, G., et al. 1993. J. Biol. Chem. 268:9194-9197. PMID: 8486620; Kiss, Z., et al. 1995. Biochim. Biophys. Acta. 1265:93-95. PMID: 7857990; Zhou, T., et al. 1999. Nat. Med. 5:42-48. PMID: 9883838, the contents of each of which is incorporated by reference herein). Contrary, ruboxistaurin is a selective and potent inhibitor of PKCβ1 and PKCβ2 (IC50=4.7 and 5.9 nM, respectively), while IC₅₀ values for other PKC isoforms α, γ, δ, ε, ζ, and η are 360, 300, 250, 600, >100,000, and 52 nM, respectively (Jirousek, M.R., Gillig, J.R., Gonzalez, C.M., et al. (S)-13-[(Dimethylamino)methyl]-10,11,14,15-tetrahydro-4,9:16,21-dimetheno-1H,13H-dibenzo[e,k]pyrrolo[3,4-h][1,4,13]oxadiazacyclohexadecene-1,3(2H)-dione (LY333531) and related analogues: Isozyme selective inhibitors of protein kinase Cβ. J Med Chem 39 2664-2671 (1996), the contents of which are incorporated by reference herein).

Non-selective PKCB inhibitors, such as Bis, may cause side effects through the inhibition of other PKC isoforms. For example, several PKC isoforms, such as η, δ, and α are thought to play a role in the regulation of keratinocyte proliferation and differentiation (e.g., Denning MF et al, Cell Growth Differ. 1995: 6(2):149-57; Kashiwagi M et al, Biochem. 2002 December; 132(6):853-7; Seo H et al, Experimental & Molecular Medicine (2004) 36, 292-299; doi:10.1038/emm.2004.40; Bollag W et al, Journal of Investigative Dermatology (2009) 129, 2330-2332. doi:10.1038/jid.2009.165; Cabodi S et al, Molecular Cell, Volume 6, Issue 5, November 2000, Pages 1121-1129). Therefore a non-selective PKC inhibitor, such as Bis, may have off target affects, including possible effects on keratinocyte proliferation, differentiation, and behavior.

An exemplary compound for use in the methods described herein is ruboxistaurin (see also U.S. Pat. Nos. 5,710,145 and 5,559,228, the entire contents of each of which is incorporated by reference herein). Ruboxistaurin has a molecular mass of 468.546 g/mol, which is a relatively large compound for local or non-systemic administration (e.g., topical administration), as compounds around or over a molecular weight of 500 g/mol are generally considered incompatible with topical delivery (Bos JD et al (2000) Exp Dermatol. 9(3):165-9). Additionally, ruboxistaurin is poorly soluble and is bright orange in color, adding further challenges to topical administration of the compound.

### Definitions

As used herein, a “hyperpigmentation condition” or “hyperpigmentation disorder” or “hyperpigmentation disease” refers to a condition characterized by general or localized increases in an individual's normal skin color. Skin hyperpigmentation disorders include for example, melasma, post-inflammatory hyperpigmentation, discoid lupus erythematous and other forms of increased epidermal melanization. A hyperpigmentation condition can also include a condition in which the pigmentation of one or more area of the skin is unwanted or otherwise characterized as excessively pigmented. A hyperpigmentation condition includes those which are mediated by melanization.

As used herein, a “selective PKCβ inhibitor” refers to a compound, which has specificity for PCK isoform β (PKCβ1 and/or PKCβ2). A selective PKCβ inhibitor has a higher specificity for PKCβ relative to at least one other PKC isoform. In an embodiment a selective PKCβ inhibitor has a higher specificity for PKCβ relative to two or more other PKC isoforms (e.g., PCKα, PKCγ). In an embodiment a selective PKCβ inhibitor has a higher specificity for PKCβ relative to, PKCα, PKCγ, PKCδ, PKCε, PKCζ, PKCι, PKCθ, and PKCη). A selective PKCβ inhibitor may inhibit activity of other non-PCKβ isoforms (e.g., PCKα); however, the inhibitory activity on non-PKCβ isoforms is high both in absolute terms and relative to the compounds inhibitory activity of PKCβ (PKCβ1 and/or PKCβ2).

As used herein, “Non-systemically” refers to a route of administration which is other than introduction into the circulatory system. Although non-systemic administration can eventually result in administered drug in the circulatory system, the administration is to other than the circulatory system.

As used herein, the terms a “patient,” “subject,” “individual,” and “host” refer to either a human or a non-human animal having or suspected of having a hyperpigmentation condition (e.g., a selective PKCβ-inhibitor responsive hyperpigmentation condition or a PKCβ mediated hyperpigmentation condition, e.g., melasma). In an embodiment the subject is a human. In an embodiment the subject is a mammal. In an embodiment the subject is a mammal other than a rodent.

As used herein, “treat” and “treating” such as a condition or disease or disorder described herein, e.g., a hyperpigmentation condition (e.g., a selective PKCβ-inhibitor responsive hyperpigmentation condition or a PKCβ mediated hyperpigmentation condition, e.g., melasma) refers to the reduction or amelioration of the progression, severity and/or duration of the condition or one or more symptoms thereof.

The term “therapeutic effect” refers to a beneficial local or systemic effect in animals, particularly mammals, and more particularly humans, caused by administration of a compound or composition described herein. The phrase “therapeutically-effective amount” or “effective amount” means that amount of a compound or composition described herein that is effective to treat a hyperpigmentation condition (e.g., a selective PKCβ-inhibitor responsive hyperpigmentation condition or a PKCB mediated hyperpigmentation condition, e.g., melasma) at a reasonable benefit/risk ratio. The therapeutically effective amount of such substance will vary depending upon the subject and disease condition being treated, the size of the lesion and condition of the skin, the age of the subject, the severity of the disease condition, the manner of administration and the like, which can readily be determined by one of skill in the art.

The term “predetermined area of skin” as used herein refers to an area of skin of a subject, e.g., a healthy area of skin, or an area of skin with a condition described herein, e.g., a hyperpigmentation condition described herein (e.g., melasma), or unwanted pigmentation. In some embodiments, a predetermined area of skin is selected, e.g., chosen or identified, based on, e.g., a measure of the amount of color, e.g., erythema or pigmentation, in the area of skin. In some embodiments, a predetermined area of skin is selected, e.g., chosen or identified, based on, e.g., a measure of the amount of color, e.g., erythema or pigmentation, in an area adjacent to the area of skin. The amount of color in skin can be measured, e.g., by a chromometer, and graded, e.g., as described herein. In some embodiments, the Individual Typology Angle (ITA) is used to grade the amount of color in skin, e.g., as described herein. In other embodiments, the Investigator Dynamic Grading Assessment (IDGA) is used to grade the amount of color in skin, e.g., as described herein.

### Selective PKC-β Inhibitors

The present disclosure features PKC-β inhibitors for use to treat a disease or disorder described herein (e.g., a hyperpigmentation condition). In some embodiments, the PKC-β inhibitor comprises a bis-indolylmaleimide, a phthalimide, or a derivative thereof. In some embodiments, the PKC-β inhibitor is a compound of Formula (I):

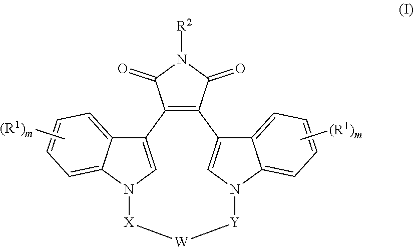

or a pharmaceutically acceptable salt, stereoisomer, racemate, or solvate thereof, wherein:
W is —O—, —S—, —S(O)—, —S(O)₂—, —C(O)—, C₂-C₆ alkylene, substituted alkylene, C₂-C₆ alkenylene, substituted alkenylene, aryl, aryl(CH₂)ₘO, heterocyclyl, heterocyclyl(CH₂)ₘO, —NR³—, —N(O)R³—, —C(O)NH—, or —NHC(O)—;
each of X and Y is independently C₁-C₄ alkylene, substituted alkylene, or X, Y, and W combine to form —(CH₂)ₙ-AA-;
each R¹ is independently hydrogen, halo, C₁-C₄ alkyl, hydroxyl, C₁-C₄ alkoxy, C₁-C₄ haloalkyl, nitro, —NR⁴R⁵, or —NHC(O)(C₁-C₄ alkyl);
R² is hydrogen, —CH₃C(O), —NH₂, or hydroxyl;
R³ is hydrogen, (CH₂)ₘaryl, C₁-C₄ alkyl, —C(O)O(C₁-C₄ alkyl), —C(O)NR⁴R⁵, —(C═NH)NH₂, —S(O)(C₁-C₄ alkyl), —S(O)₂(NR⁴R⁵), or —S(O)₂(C₁-C₄ alkyl);
each R⁴ and R⁵ is independently hydrogen, C₁-C₄ alkyl, phenyl, benzyl, or R⁴ and R⁵ are taken together with the nitrogen to which they are bonded to form a saturated or unsaturated 5- or 6-membered ring;
AA is an amino acid residue;
each m is independently 0, 1, 2, or 3; and
n is independently 2, 3, 4, or 5.

In some embodiments, X-W-Y contains 4 to 30 atoms, which may be further substituted or unsubstituted. In some embodiments, X-W-Y contains 5 atoms, 6 atoms, 7 atoms, 8 atoms, 9 atoms, 10 atoms, 11 atoms, 12 atoms, 13 atoms, 14 atoms, 15 atoms, 16 atoms, 17 atoms, 18 atoms, 19 atoms, 20 atoms, 21 atoms, 22 atoms, 23 atoms, 24 atoms, 25 atoms, 26 atoms, 27 atoms, 28 atoms, 29 atoms, or 30 atoms, which may be further substituted or unsubstituted. In some embodiments, X-W-Y contains 10 to 30 atoms, which may be further substituted or unsubstituted. In some embodiments, X-W-Y contains 20 to 30 atoms, which may be further substituted or unsubstituted.

In some embodiments, each of R¹ and R² is independently hydrogen. In some embodiments, each of X and Y is independently alkylene or substituted alkylene. In some embodiments, W is substituted alkylene, —O—, —S—, —C(O)NH—, —NHC(O)—, or NR³. In some embodiments, each of R¹ and R² is independently hydrogen, each of X and Y is independently alkylene or substituted alkylene, and W is substituted alkylene, —O—, —S—, —C(O)NH—, —NHC(O)—, or NR³.

In some embodiments, the compound of Formula (I) is a compound of Formula (Ia):

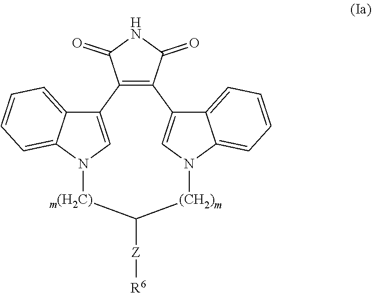

or a pharmaceutically acceptable salt, stereoisomer, racemate, or solvate thereof, wherein
Z is —(CH₂)ₚ— or —(CH₂)ₚ—O—(CH₂)ₚ;
R⁶ is hydroxyl, —SH, C₁-C₄ alkyl, (CH₂)ₘaryl, —NH(aryl), N(CH₃)(CF₃), NH(CF₃), or —NR⁴R⁵;
R⁴ is hydrogen or C₁-C₄ alkyl;
R⁵ is hydrogen, C₁-C₄ alkyl, or benzyl;
p is 0, 1, or 2; and
each m is independently 2 or 3.

In some embodiments, m is 2. In some embodiments, m is 3. In some embodiments, Z is CH₂. In some embodiments, R⁶ is NH₂, NH(CF₃), or N(CH₃)₂. In some embodiments, Z is CH₂ and R⁶ is NH₂, NH(CF₃), or N(CH₃)₂. In some embodiments, m is 2, Z is CH₂, and R⁶ is NH₂, NH(CF₃), or N(CH₃)₂.

In other embodiments, the compound of Formula (I) is a compound of Formula (Ib):

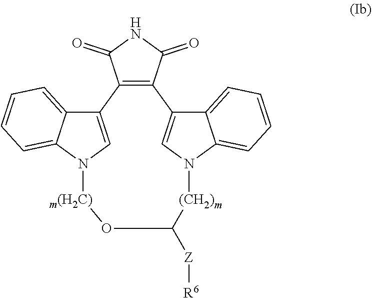

or a pharmaceutically acceptable salt, stereoisomer, racemate, or solvate thereof, wherein
Z is —(CH₂)ₚ—;
R⁶ is N(CH₃)(CF₃), NH(CF₃), or —NR⁴R⁵;
each of R⁴ and R⁵ is independently hydrogen or C₁-C₄ alkyl;
R⁵ is hydrogen, C₁-C₄ alkyl, or benzyl;
p is 0, 1, or 2; and
each m is independently 2 or 3.

In some embodiments, m is 2. In some embodiments, m is 3. In some embodiments, p is 1 or 2. In some embodiments, p is 1. In some embodiments, p is 2. In some embodiments, R⁶ is —NR⁴R⁵ and each of R⁴ and R⁵ is independently C₁-C₄ alkyl (e.g., CH₃). In some embodiments, R⁶ is N(CH₃)₂. In some embodiments, m is 2 and p is 1. In some embodiments, m is 2, p is 1, and R⁶ is N(CH₃)₂.

In other embodiments, the compound of Formula (I) is a compound of Formula (Ic):

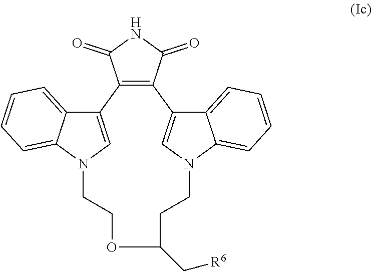

or a pharmaceutically acceptable salt, stereoisomer, racemate, or solvate thereof, wherein R⁶ is N(CH₃)(CF₃), NH(CF₃), or —NR⁴R⁵.

In some embodiments, R⁶ is —NR⁴R⁵ and each of R⁴ and R⁵ is independently C₁-C₄ alkyl (e.g., CH₃). In some embodiments, R⁶ is N(CH₃)₂.

In other embodiments, the compound of Formula (I) is a compound of Formula (Id):

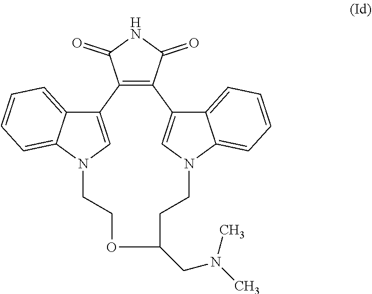 [SMILES: CN(C)CC1CCn2cc(c3ccccc32)C2=C(C(=O)NC2=O)c2cn(c3ccccc23)CCO1]

or a pharmaceutically acceptable salt, stereoisomer, racemate, or solvate thereof. In some embodiments, the compound of Formula (Id) is 9-[(dimethylamino)methyl]-6,7,10,11-tetrahydro-9H,18H-5,21:12,17-di(metheno)dibenzo[e,k]pyrrolo[3,4-h][1,2,13]oxadiazacyclohexadecine-18,20-dione, e.g., ruboxistaurin, or a pharmaceutically acceptable salt, stereoisomer, racemate, or solvate thereof.

In other embodiments, the compound of Formula (I) is a compound of Formula (Ie):

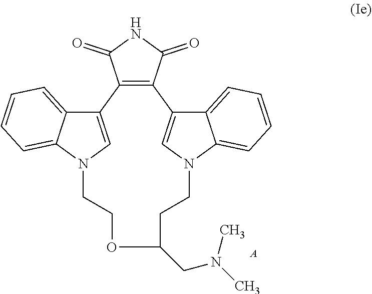

or a pharmaceutically acceptable salt, stereoisomer, racemate, or solvate thereof, wherein A is a counterion of the N(CH₃)₂ moiety. In some embodiments, the compound of Formula (Ie) is 9-[(dimethylamino)methyl]-6,7,10,11-tetrahydro-9H,18H-5,21:12,17-di(metheno)dibenzo[e,k]pyrrolo[3,4-h][1,2,13] oxadiazacyclohexadecine-18,20-dione salt, e.g., a ruboxistaurin salt, or a pharmaceutically acceptable salt, stereoisomer, racemate, or solvate thereof

In some embodiments, A is acetic acid, benzoic acid, bromine, carbonic acid, chlorine, citric acid, fluorine, fumaric acid, gluconic acid, hydrochloric acid, hydrobromic acid, hydrofluoric acid, iodine, lactic acid, phosphonic acid, phosphoric acid, methanesulfonic acid, sulfonic acid, tartaric acid, or a salt thereof. In some embodiments, A is acetic acid, benzoic acid, citric acid, hydrochloric acid, hydrobromic acid, hydrofluoric acid, methanesulfonic acid, tartaric acid, or a salt thereof. In some embodiments, A is methanesulfonic acid or a salt thereof.

In other embodiments, the compound of Formula (I) is a compound of Formula (If):

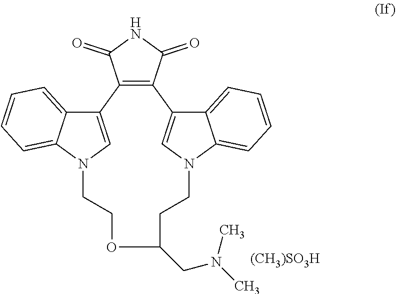

or a pharmaceutically acceptable salt, stereoisomer, racemate, or solvate thereof.

In some embodiments, the PKC-β inhibitor (e.g., a compound of Formula (If)) is 9-[(dimethylamino)methyl]-6,7,10,11-tetrahydro-9H,18H-5,21:12,17-di(metheno)dibenzo[e,k]pyrrolo[3,4-h][1,2,13]oxadiazacyclohexadecine-18,20-dione mesylate, e.g., ruboxistaurin mesylate, or a pharmaceutically acceptable salt, stereoisomer, racemate, or solvate thereof.

The compound of Formula (I) (e.g., a compound of Formulas (Ia), (Ib), (Ic), (Id), (Ie), or (If)) may exists as a solvate, e.g., a solvate with water (i.e., a hydrate), methanol, ethanol, dimethylformamide, ethyl acetate, and the like. Mixtures of solvates may also be prepared. The source of the solvate may be derived from a solvent encountered during the synthesis of the compound, e.g., the solvent of purification (e.g., crystallization) or in preparation of purification (e.g., crystallization), or adventitious to such solvent. In one embodiment, the compound of Formula (I) (e.g., a compound of Formulas (Ia), (Ib), (Ic), (Id), (Ie), or (If)) is present as a monohydrate or trihydrate solvate.

In some embodiments, the PKC-β inhibitor of Formula (I) may be a stereoisomer or racemate of the compound of Formula (I). In certain embodiments, the PKC-β inhibitor of Formula (If) is a compound of Formula (If-1) or a compound of Formula (If-2):

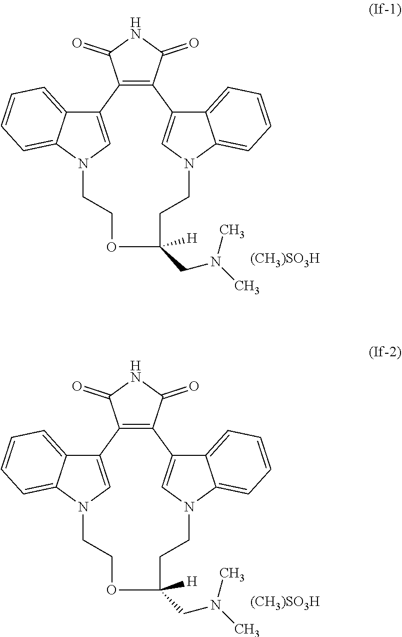

or a pharmaceutically acceptable salt, stereoisomer, racemate, or solvate thereof. In some embodiments, the PKC-β inhibitor (e.g., a compound of Formula (I)) is (9S)-9[(dimethylamino)methyl]-6,7,10,11-tetrahydro-9H,18H-5,21:12,17-di(metheno)dibenzo[e,k]pyrrolo[3,4-h][1,2,13]oxadiazacyclohexadecine-18,20-dione mesylate (e.g., Formula (If-1)) or (9R)-9[(dimethylamino)methyl]-6,7,10,11-tetrahydro-9H,18H-5,21:12,17-di (metheno)dibenzo[e,k]pyrrolo[3,4-h][1,2,13]oxadiazacyclohexadecine-18,20-dione mesylate (e.g., Formula (If-2)), e.g., ruboxistaurin mesylate, or a pharmaceutically acceptable salt, stereoisomer, racemate, or solvate thereof.

Preparation of a compound of Formula (I) (e.g., a compound of Formulas (Ia), (Ib), (Ic), (Id), (Ie), (If), (If-1), or (If-2)) may be achieved through the methods described in U.S. Pat. Nos. 5,552,396 and 6,015,807, each of which is incorporated herein by reference in its entirety. However, preparation of a compound of Formula (I) (e.g., a compound of Formulas (Ia), (Ib), (Ic), (Id), (Ie), (If), (If-1), or (If-2)) may be accomplished using other protocols or methods known to one of skill in the art.

In some embodiments, the PKC-β inhibitor is a compound of Formula (II):

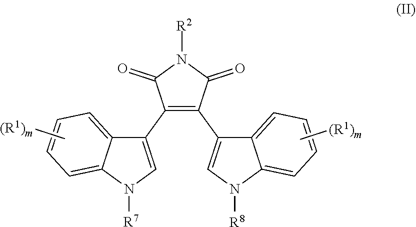

or a pharmaceutically acceptable salt, stereoisomer, racemate, or solvate thereof, wherein:
each R¹ is independently hydrogen, halo, C₁-C₄ alkyl, hydroxyl, C₁-C₄ alkoxy, haloalkyl, nitro, NR⁴R⁵, or —NHC(O)(C₁-C₄ alkyl);
R² is hydrogen, —CH₃C(O), —NH₂, or hydroxyl;
each R⁴ and R⁵ is independently hydrogen, C₁-C₄ alkyl, phenyl, benzyl, or R⁴ and R⁵ are taken together with the nitrogen to which they are bonded to form a saturated or unsaturated 5- or 6-membered ring;
R⁷ is hydrogen or C₁-C₄ alkyl;
R⁸ is cycloalkyl or heterocyclyl, each of which is substituted with one or more R⁹;
R⁹ is C₁-C₄ alkyl, C₁-C₄ alkoxy, C₁-C₄ haloalkyl, halo, cyano, nitro, aryl, arylalkyl, heteroaryl, or heteroarylalkyl, each of which is optionally substituted with one or more R¹⁰;
each R¹⁰ is independently halo, C₁-C₄ alkyl, C₁-C₄ alkoxy, C₁-C₄ haloalkyl, cyano, or nitro; and
each m is independently 0, 1, 2, or 3.

In some embodiments, each m is 0.

In some embodiments, R² is hydrogen.

In some embodiments, R⁷ is C₁-C₄ alkyl (e.g., methyl or ethyl). In some embodiments, R⁷ is methyl.

In some embodiments, R⁸ is heterocyclyl. In some embodiments, R⁸ is a nitrogen-containing heterocyclyl. In some embodiments, R⁸ is a 6-membered nitrogen-containing heterocyclyl. In some embodiments, R⁸ is piperidinyl (e.g., 1, 4-piperidinyl).

In some embodiments, R⁹ is heteroarylalkyl. In some embodiments, R⁹ is (CH2)ₙ-pyridyl, wherein n is 1, 2, 3, or 4. In some embodiments, R⁹ is (CH₂)-pyridyl. In some embodiments, R⁹ is 2-(CH₂)-pyridyl.

In some embodiments, the compound of Formula (II) is a compound of Formula (IIa):

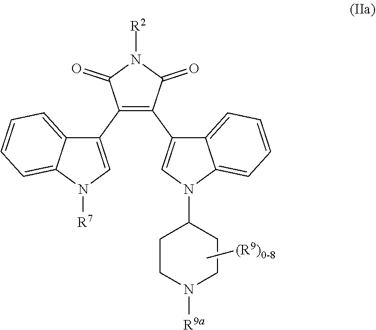

or a pharmaceutically acceptable salt, stereoisomer, racemate, or solvate thereof, wherein:
R² is hydrogen, —CH₃C(O), —NH₂, or hydroxyl;
R⁷ is hydrogen or C₁-C₄ alkyl;
R⁹ is C₁-C₄ alkyl, C₁-C₄ alkoxy, C₁-C₄ haloalkyl, halo, cyano, or nitro;
R^{9a} is aryl, arylalkyl, heteroaryl, or heteroarylalkyl, each of which is optionally substituted with one or more R¹⁰; and
each R¹⁰ is independently halo, C₁-C₄ alkyl, C₁-C₄ alkoxy, C₁-C₄ haloalkyl, cyano, or nitro.

In some embodiments, each m is 0.

In some embodiments, R² is hydrogen.

In some embodiments, R⁷ is C₁-C₄ alkyl (e.g., methyl or ethyl). In some embodiments, R⁷ is methyl.

In some embodiments, R^{9a} is heteroarylalkyl. In some embodiments, R^{9a} is (CH2)ₙ-pyridyl, wherein n is 1, 2, 3, or 4. In some embodiments, R^{9a} is (CH₂)-pyridyl. In some embodiments, R^{9a} is 2-(CH₂)-pyridyl.

In some embodiments, the compound of Formula (II) is a compound of Formula (IIb):

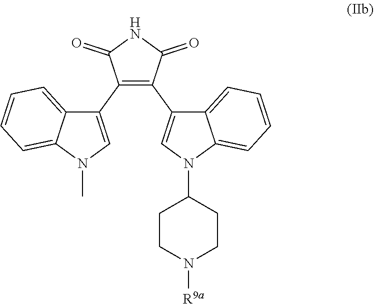

or a pharmaceutically acceptable salt, stereoisomer, racemate, or solvate thereof, wherein:
R^{9a} is aryl, arylalkyl, heteroaryl, or heteroarylalkyl, each of which is optionally substituted with one or more R¹⁰; and
each R¹⁰ is independently halo, C₁-C₄ alkyl, C₁-C₄ alkoxy, C₁-C₄ haloalkyl, cyano, or nitro; and

In some embodiments, R^{9a} is heteroarylalkyl. In some embodiments, R^{9a} is (CH2)ₙ-pyridyl, wherein n is 1, 2, 3, or 4. In some embodiments, R^{9a} is (CH₂)-pyridyl. In some embodiments, R^{9a} is 2-(CH₂)-pyridyl.

In some embodiments, the PKC-β inhibitor of Formula (II) is a compound of Formula (IIc):

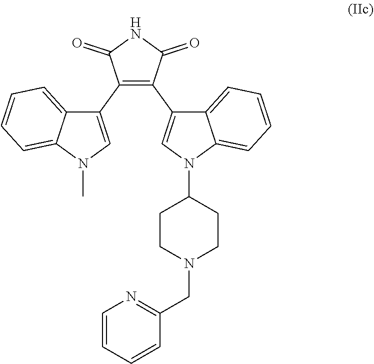 [SMILES: Cn1cc(C2=C(c3cn(C4CCN(Cc5ccccn5)CC4)c4ccccc34)C(=O)NC2=O)c2ccccc21]

or a pharmaceutically acceptable salt, stereoisomer, racemate, or solvate thereof. In some embodiments, the PKC-β inhibitor (e.g., a compound of Formula (II)) is 3-(1-methyl-1H-indol-3-yl)-4-(1-(1-(pyridin-2-ylmethyl)piperidin-4-yl)-1H-indol-3-yl)-1H-pyrrole-2,5-dione (e.g., Formula (IIc)), e.g., LY-317615 or enzastaurin, or a pharmaceutically acceptable salt, stereoisomer, racemate, or solvate thereof. In some embodiments, the PKC-β inhibitor (e.g., a compound of Formula (II)) is enzastaurin hydrochloride.

In some embodiments, the PKC-β inhibitor is a compound of Formula (III):

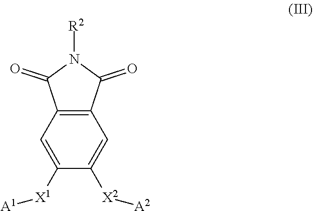

or a pharmaceutically acceptable salt, stereoisomer, racemate, or solvate thereof, wherein:
each of X¹ and X² is independently —O—, —NR⁴—, or —S—;
each of A¹ and A² is independently aryl or heteroaryl, wherein each aryl or heteroaryl is optionally substituted with one or more R⁹;
R² is hydrogen, —CH₃C(O), —NH₂, or hydroxyl;
R⁴ is hydrogen or C₁-C₄ alkyl; and
R⁹ is C₁-C₄ alkyl, C₁-C₄ alkoxy, C₁-C₄ haloalkyl, halo, cyano, or nitro.

In some embodiments, one of X¹ and X² is independently —NR⁴—. In some embodiments, each of X¹ and X² is independently —NR⁴—. In some embodiments, each of X¹ and X² is independently —NH—.

In some embodiments, one of A¹ and A² is independently aryl. In some embodiments, each of A¹ and A² is independently aryl. In some embodiments, each of A¹ and A² is independently phenyl. In some embodiments, each of A¹ and A² is independently phenyl substituted with 1 R⁹. In some embodiments, each of A¹ and A² is independently phenyl substituted with 1 R⁹ at the para position. In some embodiments, R⁹ is halo (e.g., fluoro).

In some embodiments, the PKC-β inhibitor is a compound of Formula (IIIa):

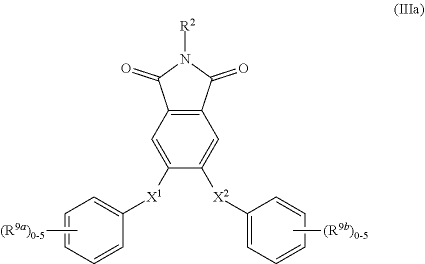

or a pharmaceutically acceptable salt, stereoisomer, racemate, or solvate thereof, wherein:
each of X¹ and X² is independently —O—, —NR⁴—, or —S—;
R² is hydrogen, —CH₃C(O), —NH₂, or hydroxyl;
R⁴ is hydrogen or C₁-C₄ alkyl; and
each of R^{9a} and R^{9b} is C₁-C₄ alkyl, C₁-C₄ alkoxy, C₁-C₄ haloalkyl, halo, cyano, or nitro.

In some embodiments, one of X¹ and X² is independently —NR⁴—. In some embodiments, each of X¹ and X² is independently —NR⁴—. In some embodiments, each of X¹ and X² is independently —NH—.

In some embodiments, each phenyl ring is substituted with 1 R^{9a} and 1 R^{9b}. In some embodiments, each phenyl ring is substituted with 1 R^{9a} and 1 R^{9b} at the para position. In some embodiments, one of R^{9a} and R^{9b} is halo (e.g., fluoro). In some embodiments, each of R^{9a} and R^{9b} is halo (e.g., fluoro).

In some embodiments, the PKC-β inhibitor of Formula (III) is a compound of Formula (IIIb):

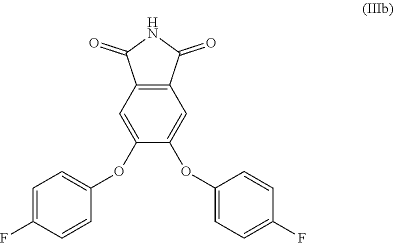 [SMILES: O=C1NC(=O)c2cc(Oc3ccc(F)cc3)c(Oc3ccc(F)cc3)cc21]

or a pharmaceutically acceptable salt, stereoisomer, racemate, or solvate thereof. In some embodiments, the PKC-β inhibitor (e.g., a compound of Formula (III)) is 5,6-bis(4-fluorophenoxy)isoindoline-1,3-dione (e.g., Formula (IIIb)), e.g., CGP 53353, CG 53353 or a pharmaceutically acceptable salt, stereoisomer, racemate, or solvate thereof.

Other PKCβ inhibitors disclosed herein include, e.g., a PKC β pseudosubstrate as described in Park et al (1999) Protein Kinase C-β Activates Tyrosinase by Phosphorylating Serine Residues in Its Cytoplasmic Domain. JBC Vol. 274, No. 23, Issue of June 4, pp. 16470-16478; and Park et al. (2004) The receptor for activated C-kinase-I (RACK-I) anchors activated PKC-β on melanosomes. Journal of Cell Science 117 (16) p. 3659. Exemplary PKC β pseudosubstrate includes e.g., an amino acid comprising the amino acid sequence Glu-Asp-Tyr-His-Ser-Leu-Tyr-Gln-Ser-His-Leu (SEQ ID NO:1), an amino acid consisting essentially of the amino acid sequence Glu-Asp-Tyr-His-Ser-Leu-Tyr-Gln-Ser-His-Leu (SEQ ID NO:1); and an amino acid consisting of the amino acid sequence Glu-Asp-Tyr-His-Ser-Leu-Tyr-Gln-Ser-His-Leu (SEQ ID NO:1). PKC β pseudosubstrates with at least 75%, 80%, 85%, 90%, 95%, 99% homologous to SEQ ID NO: 1 are also contemplated.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, 99%, or 99.9% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E.L. Stereochemistry of Carbon Compounds (McGraw-Hill, NY, 1962); Wilen, S.H. Tables of Resolving Agents and Optical Resolutions p. 268 (E.L. Eliel, Ed., Univ. of Notre Dame Press, Notre Dame, IN 1972).

As used herein, the term “alkyl” represents a saturated cyclic, straight, or branched hydrocarbon such as a straight or branched group of 1-10, 1-16, or 1-4 carbon atoms, referred to herein as C₁-C₁₀ alkyl, C₁-C₆ alkyl, and C₁-C₄ alkyl, respectively. Examples of alkyl groups include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, and the like.

As used herein, the term “alkylene” refers to the diradical of an alkyl group. Exemplary alkylene groups include methylene, ethylene, propylene, trimethylene, and the like.

As used herein, the terms “alkenyl” and “alkynyl” are art-recognized and refer to unsaturated aliphatic groups analogous in length and possible substitution to the alkyls described above, but that contain at least one double or triple bond, respectively. Exemplary alkenyl groups include, but are not limited to, —CH═CH₂ and —CH₂CH—CH₂. The terms “alkenylene” and “alkynylene” refer to the diradicals of an alkenyl and an alkynyl group, respectively.

As used herein, the term “alkoxy” refers to a straight chain or branched alkoxy group (e.g. C₁-C₆ alkyl-O—) having from one to six carbon atoms (i.e., C₁-C₆ alkoxy). Examples of alkoxy groups, include, but are not limited to, groups such as methoxy, ethoxy, propyloxy, isopropyloxy, butyloxy, isobutyloxy, tert-butyloxy, pentyloxy, or hexyloxy, and the like.

As used herein, the term “aryl” refers to a monocyclic, bicyclic or polycyclic hydrocarbon ring system, wherein at least one ring is aromatic. Representative aryl groups include fully aromatic ring systems, such as phenyl, naphthyl, and anthracenyl, and ring systems where an aromatic carbon ring is fused to one or more non-aromatic carbon rings, such as indanyl, phthalimidyl, naphthimidyl, or tetrahydronaphthyl, and the like.

As used herein, the term “cycloalkyl” refers to a monocyclic, or fused, spiro-fused, and/or bridged bicyclic or polycyclic hydrocarbon ring system, wherein each ring is either completely saturated or contains one or more units of unsaturation, but where no ring is aromatic. Representative cycloalkyl groups include cyclopentyl, cyclobutyl, cyclopentyl, cyclohexyl, and the like.

As used herein, the terms “halo” and “halogen” refer to fluorine (fluoro, —F), chlorine (chloro, —Cl), bromine (bromo, —Br), or iodine (iodo, —I).

As used herein, the term “haloalkyl” refers to an alkyl radical substituted with one or more halogen groups, e.g., CF₃.

As used herein, the terms “hydroxyl” and “hydroxyl” refer to a —OH radical.

As used herein, the term “heteroaryl” refers to a monocyclic, bicyclic or polycyclic ring system wherein at least one ring is both aromatic and comprises a heteroatom; and wherein no other rings are heterocyclyl. Representative heteroaryl groups include ring systems where (i) each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl; (ii) each ring is aromatic or cycloalkyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3(4H)-one, 5,6,7,8-tetrahydroquinolinyl and 5,6,7,8-tetrahydroisoquinolinyl; and (iii) each ring is aromatic or cycloalkyl, and at least one aromatic ring shares a bridgehead heteroatom with another aromatic ring, e.g., 4H-quinolizinyl.

In certain embodiments, the heteroaryl is a monocyclic or bicyclic ring, wherein each of said rings contains 5 or 6 ring atoms where 1, 2, 3, or 4 of said ring atoms are a heteroatom independently selected from N, O, and S.

As used herein, the terms “arylalkyl” and “heteroarylalkyl” refer to an (aryl)alkyl- or (heteroaryl)alkyl-radical, respectively, wherein aryl and heteroaryl are as described herein.

As used herein, the term “heterocyclyl” refers to a monocyclic, or fused, spiro-fused, and/or bridged bicyclic and polycyclic ring system where at least one ring is saturated or partially unsaturated (but not aromatic) and comprises a heteroatom. Representative heterocyclyls include ring systems in which (i) every ring is non-aromatic and at least one ring comprises a heteroatom, e.g., tetrahydrofuranyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl; (ii) at least one ring is non-aromatic and comprises a heteroatom and at least one other ring is an aromatic carbon ring, e.g., 1,2,3,4-tetrahydroquinolinyl, 1,2,3,4-tetrahydroisoquinolinyl; and (iii) at least one ring is non-aromatic and comprises a heteroatom and at least one other ring is aromatic and comprises a heteroatom, e.g., 3,4-dihydro-1H-pyrano[4,3-c]pyridine, and 1,2,3,4-tetrahydro-2,6-naphthyridine. In certain embodiments, the heterocyclyl is a monocyclic or bicyclic ring, wherein each of said rings contains 3-7 ring atoms where 1, 2, 3, or 4 of said ring atoms are a heteroatom independently selected from N, O, and S.

As used herein, the term “nitro” refers to a —NO₂ radical.

As described herein, compounds of the invention may contain “substituted” moieties. In general, the term “substituted”, whether preceded by the term “optionally” or not, means that one or more hydrogens of the designated moiety are replaced with a suitable substituent. Unless otherwise indicated, a “substituted” group may have a suitable substituent at each substitutable position of the group, and when more than one position in any given structure may be substituted with more than one substituent selected from a specified group, the substituent may be either the same or different at each position. In a broad aspect, the permissible substituents include acyclic and cyclic, branched and unbranched, carbocyclyl and heterocyclyl, aromatic and nonaromatic substituents of organic compounds (e.g., alkyl, alkenyl, alkynyl, carbocyclyl, heterocyclyl, aryl, or heteroaryl, any of which may itself be further substituted), as well as halogen, carbonyl (e.g., aldehyde, ketone, ester, carboxyl, or formyl), thiocarbonyl (e.g., thioester, thiocarboxylate, or thioformate), amino, —N(R^{b})(R^{c}), wherein each R^{b} and R^{c} is independently H or C₁-C₆ alkyl, cyano, nitro, —SO₂N(R^{b})(R^{c}), —SOR^{d}, and S(O)₂R^{d}, wherein each R^{b}, R^{c}, and R^{d} is independently H or C₁-C₆ alkyl. Illustrative substituents include, for example, those described herein above.

The permissible substituents can be one or more and the same or different for appropriate organic compounds. This disclosure is not intended to be limited in any manner by the permissible substituents of organic compounds. It will be further understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

### Pharmaceutical Compositions

Pharmaceutical compositions comprise one or more compounds disclosed herein and one or more physiologically or pharmaceutically acceptable carrier. The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The pharmaceutical compositions of the disclosure may also be administered locally or non-systemically, e.g., topically, or intradermally, especially when the target of treatment includes areas or organs readily accessible by local or non-systemic application, including the skin. Suitable local or non-systemic formulations are readily prepared for administration to the skin. The pharmaceutical composition cam be formulated as a lotion, cream, serum, spray, mousse, aerosol, emulsion, cake, ointment, gel, paste, patch, pencil, towelette, mask, stick, foam, elixir, or concentrate. The pharmaceutical composition can be formulated to include an agent selected from: a lathering surfactant, a vitamin, a hydroxy acid, a sunscreen, an antioxidant, a retinoid, and a moisturizing agent.

### Exemplary Topical and Cosmetic Pharmaceutical Compositions

Pharmaceutical compositions described herein may be formulated for topical administration. In some embodiments, the topical formulation is an emulsion, cream, lotion, gel, oil, ointment, aerosol spray, or semi-solid formulation. In some embodiments, the topical formulation comprises a carrier, wherein said carrier is selected from the group consisting of trehalose, malto-dextrin, rice flour, micro-crystalline cellulose, magnesium stearate, inositol, fructo-oligosaccharide, gluco-oligosaccharide, dextrose, sucrose, talc, water, physiological salt solution, urea, methanol, ethanol, propanol, butanol, ethylene glycol, propylene glycol, white pertrolatum, isopropyl myristate, lanolin, lanolin alcohol, mineral oil, lavender oil, nasturtium extract oil, sorbitan mono-oleate, cetylstearyl alcohol, hydroxypropyl cellulose, detergent, sucrose stearate, sucrose cocoate, sucrose distearate, 2-ethyl-1,3-hexanediol, polyoxypropylene-15-stearyl ether, glycerol stearate, glycerin, synthetic spermaceti, cetyl alcohol, butylparaben, propylparaben, and methylparaben.

Pharmaceutical compositions described herein may be formulated as cosmetic compositions. The cosmetic compositions can comprise a dermatologically acceptable vehicle. The dermatologically acceptable vehicle can comprise water, mineral oil, petrolatum, ceresin, lanolin alcohol, methylchloroisothiazolinone, and methylisothiazolinone. Examples of emollients that can be used as suitable dermatological carriers include, but are not limited to stearyl alcohol, glycerol monoricinoleate, glycerol monostearate, mink oil, cetyl alcohol, isopropyl isostearate, stearic acid, isobutyl palmitate, isocetyl stearate, oleyl alcohol, isopropyl luarate, hexyl laurate, decyl oleate, octadecan-2-ol, isocetyl alcohol, eicosanylalcohol, behznyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, tallow, lard, olive oil, palm kernal oil, rapeseed oil, safflower seed oil, evening primrose oil, soybean oil, sunflower seed oil, avocado oil, olive oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, and myristyl myristate. More than one emollient may be included in the composition. The dermatological acceptable vehicle can be present in an amount of from about 10 wt % to about 99.999 wt %, for example about 20 wt % to about 99.999 wt %, about 40 wt % to about 90 wt % of the cosmetic composition. In some aspects, the cosmetic composition can also comprise water up to about 98% volume, for example, about 5 to about 80%>volume of the cosmetic composition.

Examples of propellants that can be used as suitable dermatological carriers include, but are not limited to, a liquefiable gas or a halogenated propellant. Specific examples of propellants include dimethyl ether, trichlorofluoromethane, diclorodifluoromethane, dichlrorotetrafluroethane, monochlorodif uoromethane, trichlorotrifluoroethane, propane, butane, carbon dioxide, nitrous oxide, or combinations thereof. The amount of propellant in the composition can be from about 10% to about 60% by weight of the composition.

Examples of solvents that can be used as suitable dermatological carriers include, but are not limited to ethyl alcohol, methylene chloride, isopropanol, acetone, ethylene glycol monoethyl ether, diethylene glycol monobutyl ether, diethylene glycol monoethyl ether, dimethyl sulphoxide, dimethyl formamide, tetrahydrofuran, propylene glycol, butylene glycol, dipropylene glycol, ethoxylated or propoxylated diglycols, cyclic polyols, or combinations thereof.

Examples of emulsifiers that can be used as suitable dermatological carriers include nonionic, anionic or cationic emulsifiers. Suitable emulsifiers are disclosed, for example, in McCutcheon's Detergents and Emulsifiers, North American Edition, pp. 317-324 (1986), and the ICI Handbook, pp. 1673-1686. Single emulsion skin care preparations, such as lotions and creams, of the oil-in-water type and water-in-oil type are well known in the cosmetic art and are useful in the present disclosure. Multiphase emulsion compositions, for example the water-in-oil-in-water type, as disclosed in U.S. Pat. Nos. 4,254,105 and 4,960,764, which are incorporated by reference herein in their entireties for their teachings of emulsions, can also be useful. In general, such single or multiphase emulsions contain water, emollients, and emulsifiers as essential ingredients. The compositions can comprise from about 1%>to about 10%> (e.g., from about 2%>to about 5% >) of an emulsifier(s). The emulsion can be a microemulsion or a nanoemulsion.

Examples of powders that can be used as suitable dermatological carriers include, but are not limited to chalk, talc, fullers earth, kaolin, starch, gums, colloidal silica sodium polacrylate, tetre alkyl and/or trialkyl aryl ammonium smectites, chemically modified magnesium aluminium silicate, organically modified montmorillonite clay, hydrated aluminium silicate, fumed silica, carboxyvinyl polmer, sodium carboxymethyl cellulose, ethylene glycol monostearate, or combinations thereof.

The cosmetic compositions described herein can contain additional oil-soluble materials and/or water-soluble materials conventionally used in compositions for use on skin, hair, and nails. Specific examples of other suitable agents that can be included in the cosmetic composition, include a moisturizing agent, humectants, surface active agents, binding agents, thickeners, viscosity modifiers, buffers, preservatives, neutral or cationic lipids, lipid complexes, liposomes, polymers, sunscreen agents, lubricants, antioxidants, proteins, amino acids, fragrances, perfumes, oils, natural extracts such as plant extracts, butters, vitamins, pH adjusting agents, absorbents, other dermatological acceptable excipients, and combinations thereof.

The cosmetic compositions can comprise a moisturizing agent or a humectant. In some examples, the cosmetic composition can be combined with other ingredients such as moisturizers, cosmetic adjuvants, anti-oxidants, depigmenting agents, darkening agents, anti-aging agents, hair removal agents, hair styling agents, nail styling agents, sunscreens, surfactants, bleaching agents, foaming agents, conditioners, humectants, fragrances, colorants, viscosifiers, buffering agents, preservatives, and the like and mixtures thereof. Skin-care compositions including these components should be formulated so as not to affect the activity of the compound, i.e. a compound disclosed herein.

Examples of humectants include glycerol, sorbitol, propylene glycol, ethylene glycol, 1,3-butylene glycol, polypropylene glycol, xylitol, malitol, lactitol, allantoin, acetamine MEA, oat protein, hyaluronic acid, and the like. They can be used either singly or in combination. Preservatives can also be included in the cosmetic compositions.

Preservatives are useful for substantially preventing microbial decomposition. Examples of suitable preservatives include phenoxyethanol and parabens such as methyl-paraben, ethyl-paraben, and propyl-paraben; salicylic acid, chlorhexidine hydrochloride, phenoxyethanol, sodium benzoate, methyl para-hydroxybenzoate, ethyl para-hydroxybenzoate, propyl para-hydroxybenzoate, butyl para-hydroxybenzoate, isothiazolones and the like. Other examples of preservatives are listed on pages 1654-55 of the International Cosmetic Ingredient Dictionary and Handbook, eds. Wenninger and McEwen (CTFA, 7th ed., 1997), hereinafter referred to as the “Cosmetic Handbook.” The composition can comprise from about 0.01% to about 20%, by weight (more preferably, from about 0.5%>to about 5%, by weight) of preservative. Microbial contamination can also be eliminated by gamma irradiation or microfiltration, or by brief heat treatments that do not result in the elimination of protease inhibitory activity.

Examples of fragrances and odor masks that can be included in the disclosed compositions include menthol, anethole, carvone, eugenol, limonene, ocimene, n-decylalcohol, citronellol, a-terpineol, methyl salicylate, methyl acetate, citronellyl acetate, cineole, linalool, ethyl linalool, vanillin, thymol, spearmint oil, peppermint oil, lemon oil, orange oil, sage oil, rosemary oil, cinnamon oil, pimento oil, cinnamon leaf oil, perilla oil, wintergreen oil, clove oil, eucalyptus oil and the like.

Examples of surface active agents that can be included in the disclosed compositions include sodium alkyl sulfates, e.g., sodium lauryl sulfate and sodium myristyl sulfate, sodium N-acyl sarcosinates, e.g., sodium N-lauroyl sarcosinate and sodium N-myristoyl sarcosinate, sodium dodecylbenzenesulfonate, sodium hydrogenated coconut fatty acid monoglyceride sulfate, sodium lauryl sulfoacetate and N-acyl glutamates, e.g., N-palmitoyl glutamate, N-methylacyltaurin sodium salt, N-methylacylalanine sodium salt, sodium a-olefin sulfonate and sodium dioctylsulfosuccinate; N-alkylaminoglycerols, e.g., N-lauryldiaminoethylglycerol and N-myristyldiaminoethylglycerol, N-alkyl-N-carboxymethylammonium betaine and sodium 2-alkyl-1-hydroxy ethylimidazoline betaine; polyoxyethylenealkyl ether, polyoxyethylenealkylaryl ether, polyoxyethylenelanolin alcohol, polyoxyethyleneglyceryl monoaliphatic acid ester, polyoxyethylenesorbitol aliphatic acid ester, polyoxyethylene aliphatic acid ester, higher aliphatic acid glycerol ester, sorbitan aliphatic acid ester, PIURONIC™ type surface active agent, and polyoxyethylenesorbitan aliphatic acid esters such as polyoxyethylenesorbitan monooleate, polyoxyethylenesorbitan monolaurate, and combinations thereof.

Examples of the binder or thickener include cellulose derivatives such as alkali metal salts of carboxymethylcellulose, methyl cellulose, hydroxyethyl cellulose and sodium carboxymethylhydroxyethyl cellulose, alkali metal alginates such as sodium alginate, propylene glycol alginate, gums such as carrageenan, xanthan gum, tragacanth gum, caraya gum and gum arabic, and synthetic binders such as polyvinyl alcohol, polysodium acrylate and polyvinyl pyrrolidone. Thickening agents that can be added to the compositions to alter viscosity include other polymers such as polyacrylates (e.g., polyacrylamide). Other examples of viscosity modifying agents are listed on pages 1692-97 of the Cosmetic Handbook, which is incorporated by reference herein in its entirety for its teachings of viscosity modifying agents. To achieve the appropriate viscosity, the cosmetic compositions may comprise from about 0.01% to about 20%, by weight (e.g., from about 0.1% >to about 5%, by weight) of a thickening agent.

Coloring agents and fragrances can also be included in the compositions comprising compounds disclosed herein. The dermatological acceptable vehicle can include an emollient, a diluent, a solubilizing or emulsifying agent, a solvent, a propellant, a powder, or combinations thereof. The cosmetic compositions can also comprise a moisturizing agent or a humectant. In some embodiments, the cosmetic composition is an emulsion, cream, lotion, gel, oil, ointment, aerosol spray, or semi-solid formulation.

### Routes of Administration

Administration of a selective PKCβ inhibitor described herein to a subject may be local or non-systemic, e.g., topical, intradermal, subcutaneous. In an embodiment, a selective PKCβ inhibitor described herein is topically administered. In an embodiment, a selective PKCβ inhibitor described herein is intradermally administered. In an embodiment, a selective PKCβ inhibitor described herein is subcutaneously administered. In some embodiments, a selective PKCβ inhibitor described herein is administered through contacting a patch (comprising said selective PKCβ inhibitor) to the skin (e.g., an area of the skin affected by the hyperpigmentation disorder or an area of the skin surrounding an area of the skin affected by the hyperpigmentation disorder).

In an embodiment, the method comprises applying the selective PKCβ inhibitor to a predetermined region of the body, e.g., a predetermined region of skin, e.g., as described herein. In an embodiment, the method comprises applying the selective PKCβ inhibitor to an area of the skin affected by the hyperpigmentation condition. In an embodiment, the method comprises applying the selective PKCβ inhibitor to an area adjacent to an area of the skin affected by the hyperpigmentation condition. In an embodiment, the method comprises applying the selective PKCβ inhibitor to the face, arms, back, or other areas affected by unwanted or otherwise excess hyperpigmentation.

### Dosages

Toxicity and therapeutic efficacy of the selective PKCβ inhibitors, e.g., a selective PKCβ inhibitor described herein, can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected skin in order to minimize potential damage to uninfected cells and, thereby, reduce side effects.

For any compound, the therapeutically effective dose can be estimated initially from cell culture assays (e.g., melanocyte cell culture assays, e.g., melanoma cells). A dose may be formulated in animal models to achieve a level in the skin that includes the IC₅₀ (i.e., the concentration of the test compound that achieves a half-maximal inhibition of symptoms) as determined in cell culture. Such information can be used to more accurately determine useful doses in humans. In addition, levels in plasma may be measured, for example, by high performance liquid chromatography, in order to ascertain systemic exposure.

It should also be understood that a specific dosage and treatment regimen for any particular patient will depend upon a variety of factors, including the activity of the specific compound employed, the age, the size of the lesion, number of lesions, general health, sex, diet, time of administration, drug combination, and the judgment of the treating physician and the severity of the particular disease being treated. The amount of a selective PKCβ inhibitor, e.g., a selective PKCβ inhibitor described herein, in the composition will also depend upon the particular selective PKCβ inhibitor in the composition.

### Methods of Treatment

In another aspect, the disclosure provides methods of treating a subject having a hyperpigmentation disorder (e.g., melasma), the method comprising administering, e.g., locally or non-systemically, e.g., topically, or intradermally, an effective amount of a selective PKCβ inhibitor, e.g., a selective PKCβ inhibitor described herein, e.g., ruboxistaurin, to the subject, thereby treating the subject.

In an embodiment, the method comprises administering the selective PKCβ inhibitor to the subject, daily, every other day, weekly, or monthly. In an embodiment, the method comprises administering the selective PKCβ inhibitor to the subject about twice daily, about once daily, about every two days, about every three days, about every four days, about every five days, about every six days, about once a week, about every other week, e.g., biweekly, about once a month, e.g., monthly. In an embodiment, the subject has received one or more previous administrations of the compound, e.g., at least 2, 10, 20, 30, 40, 50, 100, 200, 300, or 500 previous administrations of the compound. In an embodiment, the subject has previously been treated with the compound, e.g., had been treated for at least 1, 6, 12, 24, 36, or 48 months. In an embodiment, the method comprises one or more subsequent administrations of the selective PKCβ inhibitor, e.g., at least 2, 3, 4, 5, 6, 7, 8, 9, 10, 12, 15, 20, 50 or 100 administrations are administered to the subject. In an embodiment, the method comprises one or more subsequent administrations of the selective PKCB inhibitor, e.g., at least 2, 3, 4, 5, 6, 7, 8, 9, 10, 12, 15, 20, 50 or 100 administrations are administered to the subject. In an embodiment, the method comprises administering the selective PKCB inhibitor to the subject about every hour, about every 2 hours, about every 3 hours, about every four hours, e.g., about 3 to 4 times per day.

### Hyperpigmentation Disorders

The selective PKCB inhibitors described herein can be used to treat hyperpigmentation disorders, e.g., skin hyperpigmentation disorder, e.g., melasma. In one embodiment the hyperpigmentation disorder is melasma. In one embodiment the hyperpigmentation disorder is post inflammatory hyperpigmentation. In one embodiment the hyperpigmentation disorder is lentigines (e.g., age spots). In one embodiment, the hyperpigmentation disorder is associated with ultra-violet (UV) radiation exposure, e.g., sun exposure or a tanning response. In one embodiment the hyperpigmentation disorder is a tanning response. In one embodiment the hyperpigmentation condition is chosen from: melasma, post inflammatory hyperpigmentation, discoid lupus erthyematous, phytophotodermatitis, lentigines (e.g., age spots), birth marks, café au lait macules, acanthosis nigricans, burn associated hyperpigmentation, lentigenes, drug-induced hyperpigmentation (e.g., sulfonamide, tetracycline, NSAID, barbiturate, and carbamazepine induced hyperpigmentation), injury induced hyperpigmentation, primary biliary cirrhosis associated hyperpigmentation, Addison's disease associated hyperpigmentation, melanocytic naevi, ephelides, seborrhoeic keratosis, skin cancer associated hyperpigmentation, infection associated hyperpigmentation (e.g., pityriasis versicolor, erythrasma), eczema, photocontact dermatitis, ichthyosis, neurofibromatosis, or hyperpigmentation associated with ultra-violet (UV) radiation exposure, e.g., sun exposure or a tanning response.

In an embodiment the disorder is other than an age-related disorder.

### Combination Treatments

The selective PKCβ inhibitors described herein can be administered in combination with one or more additional agents, e.g., therapeutic agents. The one or more additional agent can include, but is not limited to, hydroquinone, tretinoin, a corticosteroid, azaleic acid, kojic acid, a retinoid, glycolic acid, L-ascorbic acid, sunscreen, p-aminobenzoic acid, padimate O, phenylbenzimidazole sulfonic acid, cinoxate, menthyl anthranilate, dioxybenzone, oxybenzone, avobenzone, octisalate, octocrylene, octyl methoxycinnamate, homosalate, octinoxate, sulisobenzone, trolamine salicylate, ecamsule, zinc oxide and titanium dioxide. The selective PKCβ inhibitors described herein can be administered in combination with prior fraxel laser therapy, tape stripping or chemical removal of the stratum corner barrier. These treatments may enhance the penetration of the selective PKCβ inhibitors described herein.

### EXAMPLES

The following examples are intended to be illustrative, and are not meant in any way to be limiting.

### Example 1. Effectiveness of the Selective PKCβ Inhibitor Ruboxistaurin in Lightening Pigmentation

Previous work has shown that the non-selective PKC inhibitor Bisindolylmaleimide (Bis) decreases the activity of tyrosinase, the rate limiting enzyme in melanogenesis, by preventing its phosphorylation by PKC-β (e.g., Park et al. (2004) J of Investigative Dermatology 122: 159-166; U.S. Pat. No. 5,962,417). Because other PKC isoforms play multiple roles in the skin, while PKC-β appears to function exclusively in the skin as an activator of tyrosinase, a drug to lighten skin would preferably (e.g., for, inter alia, safety and to decrease side effects) selectively inhibit the β isoform. The following example demonstrates the effectiveness of the selective PKCβ inhibitor ruboxistaurin in lightening pigmentation.

Briefly, C47 BI/6 mice aged 8-9 weeks (3 per group) were depilated to remove back hairs. These mice have black hair but no melanin pigmentation in the skin. Melanocytes in the hair follicle are responsible for the coat color. Topical formulations (DMSO: Propylene glycol/25:75) made with 4% hydroquinone (HQ), 100 μm (0.0057%) Bis, or 100 μm (0.0057%) ruboxistaurin were prepared for administration. All compounds and vehicle were applied daily to the depilated areas for 19 days after depilation, i.e. throughout the period during which the new hair shafts are produced following depilation. Representative hairs of each type: guard, awl (large follicles deep in the dermis or subcutaneous follicle), auchene, and zig-zag (smaller follicles set in the more superficial dermis and the most numerous type of coat hairs), were randomly plucked (50/mouse) and photographed under a dissecting microscope. For separate analysis, 20 dark and light hairs from each mouse were subjected to computer assisted analysis of “gray value” for the pixels comprising the hair shaft.

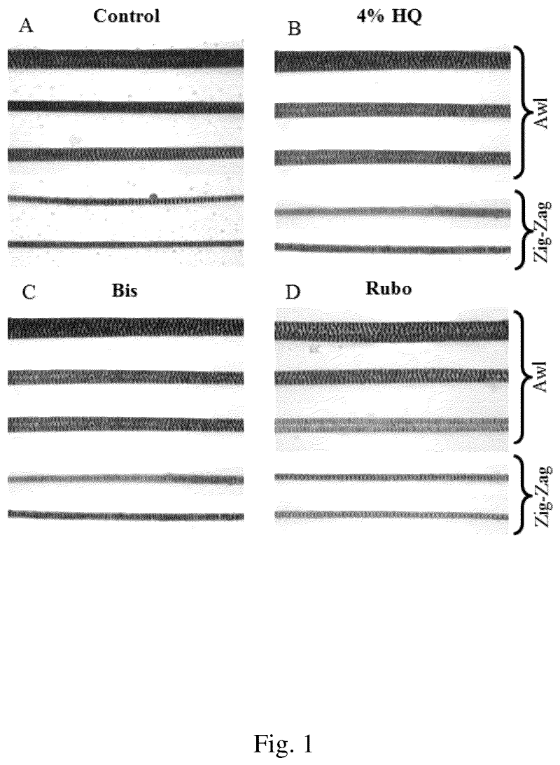

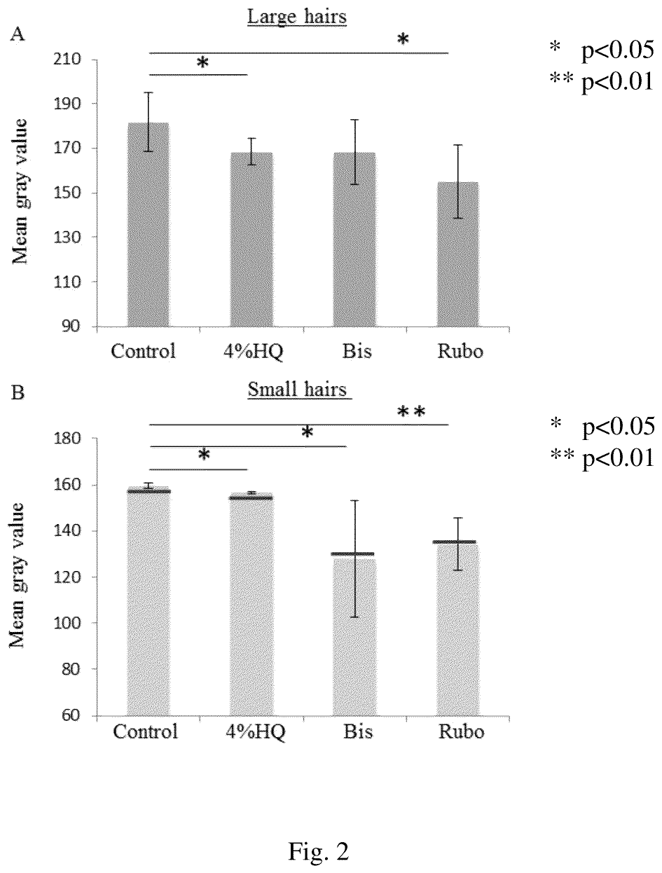

As shown in FIG. 1, awl hairs showed a modest difference in color with little color variation within each group, and all experimental groups appeared lighter than control group (FIGS. 1A and B). Zig-zag hairs treated with 4% HQ showed minimal color difference compared to vehicle control, whereas both Bis and ruboxistaurin treated hairs were significantly lighter and quite variable in color (FIGS. 1A, C, and D). Average gray value within the hair shafts were determined using ImageJ (NIH) software. The gray value is the sum of the gray values of all the pixels in the hair shaft divided by the number of pixels; and values range from 0 (white, no pigment) to 255 (black). Awl hairs showed average reductions in gray value for 4% HQ of 168, Bis of 168, and ruboxistaurin of 155 compared to the control value of 181. Zig-zag hairs also showed significant average reductions of gray value in all treated groups with 4% HQ: 156; Bis: 127; and ruboxistaurin: 134; compared to the control: 159 (FIG. 2).

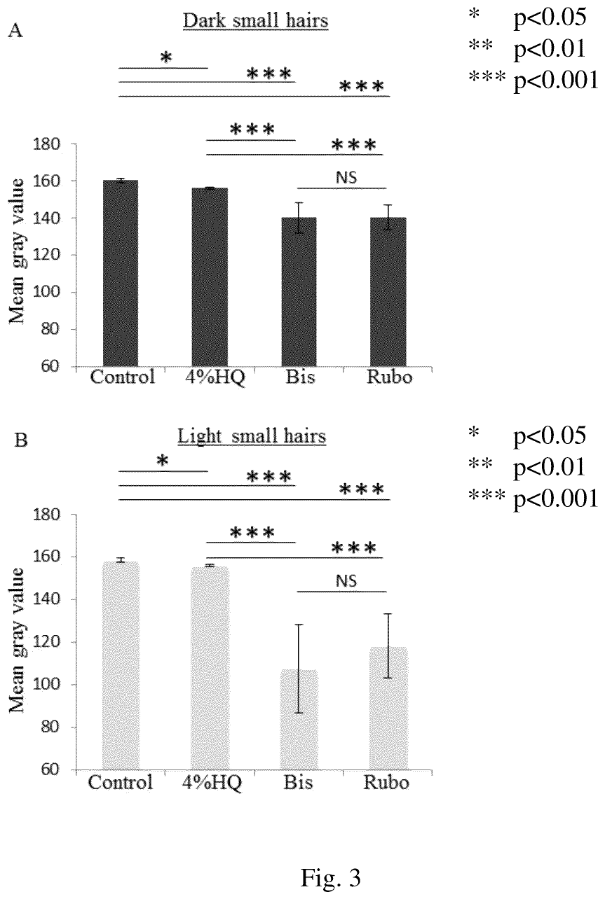

To determine the maximum degree of hair lightening (attributable to maximal drug exposure), an additional comparison was made between zig-zag hairs in each group that fell below versus above the median gray value. This analysis resulted in less variation for all groups (smaller standard deviation) and highly statistically significant differences among the groups. 4% HQ treated hairs were minimally lighter than control hairs, whereas ruboxistaurin hairs were lighter than both control and 4% HQ hairs (FIG. 3). Overall, the in vivo experimental data described herein show that ruboxistaurin is not only an effective lightening agent but as a lightening agent is significantly more potent (e.g., at a 0.0057% concentration) than HQ (e.g., at a 4.0% concentration (a standard concentration used in current prescription lightening products).

### Example 2: Clinical Trial Evaluating Ruboxistaurin Gel in Ultraviolet Light-Induced Melanogenesis in Normal Volunteers

A clinical trial was performed to evaluate the ability of a gel formulation of ruboxistaurin or placebo to inhibit ultraviolet (UV) light-induced melanogenesis in normal volunteers. The trial was observer-blinded and placebo controlled. The formulations of ruboxistaurin used in the trial included 0.1%, 0.5% and 1.0% ruboxistaurin gel. One of the objectives of this trial was to determine whether topical administration of ruboxistaurin gel can inhibit ultraviolet light induced melanogenesis.

### Trial Design

There were 5 treatment sites in each subject. Four sites were administered ruboxistaurin gel or vehicle, and one site was left untreated. The formulations of ruboxistaurin used in the trial were 0.1%, 0.5% and 1.0% ruboxistaurin gel. Subjects, e.g., normal volunteers, were administered a 3 minimal erythema dose (MED) of UV radiation, i.e., a UV dose expected to produce erythema, e.g., sunburn, followed by development of a tan several days later, roughly equivalent to a 1-2 hour mid-day spring sun exposure. Each UV irradiation was done at a minimum erythema dose (MED). Following 3 doses of MED UV irradiation, subjects were administered ruboxistaurin gel or vehicle at four sites. All five sites, including four sites treated and one UV irradiated but untreated site, were under occlusion for 15 days.

The predetermined primary endpoint for this study was Individual Typology Angle (ITA) colorimeter readings obtained on day 24, when all erythema, e.g., sunburn, was expected to have disappeared. To quantitatively measure color in skin, e.g., erythema or pigmentation, a chromometer was used. Pigmentation was measured with the Individual Typology Angle (ITA), wherein a lower number indicated a darker, e.g., more deeply tanned, test area.

Secondary endpoints of the study included ITA colorimeter readings at days 7, 14, and 17; Investigator Dynamic Grading Assessment (IDGA) scores at days 7, 14, 17, and 24; and biopsies for melanin on day 24. The Investigator Dynamic Grading Assessment (IDGA) score is a measure used to assess color in skin by a blinded observer, wherein a higher number indicated a darker test area as shown in Table 1.

TABLE 1
IDGA assessment scale

| IDGA | Description of Pigmentation |
|---|---|
| −2 | Much less pigmentation than the control site |
| −1 | Slightly less pigmentation than control site |
| 0 | Similar pigmentation to the UV irradiated but untreated |
| | control site |
| 1 | Slightly darker than control site |
| 2 | Moderately darker than control site |
| 3 | Markedly darker than control site |

On Day 1, all subjects enrolled in the study were examined for uneven skin tones and blemishes, using a Wood's lamp at the following sites: the back, between the belt-line and shoulder blades. For MED dose administration, a timed series of 6 UV doses, increasing in 25% increments, were administered to the left side of the mid-back using a multiport solar simulator.

Duoderm dressing was applied to on the right side of the back. Test areas were graded for pigmentation, erythema, and colorimeter and readings were obtained. Five test articles, i.e., ruboxistaurin gel, were applied to each subject. Subjects were then instructed to avoid UV exposure, photosensitizers, antihistamines, anti-inflammatory medications and other prohibited medications and to return to the testing laboratory 16 to 24 hours after completion of UV doses to determine the MED doses of each subject.

Ruboxistaurin gel was applied in a metal test chamber (12 mm inside Ø, 14 mm outside Ø), seated in holes punched in a hydrocolloid dressing (DuoDerm). Approximately 200 μl of the 5 test articles were pipetted on filter paper placed in the test chambers. The Duoderm dressing with 6×15 mm holes spaced at least 1.5 cm apart were fixed to the skin with adhesive tape containing the same holes. The chambers themselves were fixed in place with adhesive tape and were removed before each new application. Before each new application remaining preparation residues were removed by gently cleansing each test field with a separate soft tissue. The hydrocolloid dressing stayed in place until the sites were evaluated on Days 7 and 14 and 17 but were renewed more frequently when necessary. In case of a missed day of treatment/evaluation, the subject was advised to keep the chamber and patches in place until he/she could return to the clinical center. The subject returned to the clinical center at the next scheduled dosing occasion.

On Day 2, a trained evaluator graded the responses of the UV exposed sites, under warm fluorescent or tungsten illumination of at least 450 lux. Grading scales for erythema responses to UV Doses were as indicated in Table 2. MED is defined as the first exposure site in the series that produced an erythema grade of at least 2. On the right side of the back, a UV dose of 3 MEDs was administered to each of the 5 sites. Immediately after the UV doses were administered the ruboxistaurin gel was applied.

TABLE 2
Erythema grading

| Grading | Response |
|---|---|
| 0 | No erythema response |
| 1 | Minimally perceptible erythema |
| 2 | Perceptible erythema with clearly defined borders |
| 3 | Moderate erythema with sharp borders |
| 4 | Dark red erythema with sharp borders |
| 5 | Dark red erythema with sharp borders and possible |
| | edema* |
| 6 | Intense erythema with sharp borders and edema |

On days 3, 4, 5, 6, 8, 9, 10, 11, 12, 13, 15, 16, the previously applied ruboxistaurin gel was removed. The test sites were evaluated for signs and symptoms of irritation, following which the ruboxistaurin gel was reapplied.

On days 7, 14, 17, and 24, the previously applied ruboxistaurin gel was removed and the Duoderm dressing was removed. After 15 minutes, the test sites were evaluated for signs and symptoms of irritation. Erythema and pigment darkening were then graded, and colorimetric measurements were taken. Crossed polarized and non-polarized digital images of each test site were taken. Duoderm dressing and ruboxistaurin gel was reapplied on days 7 and 14. No ruboxistaurin gel was applied on Days 17 and 24. Skin biopsies were obtained from the 5 test sites after all measurements were obtained on Day 24.

### Results

ITA readings taken from all subjects demonstrated that the majority of test areas, e.g., areas in which ruboxistaurin gel was applied, were lighter than placebo on Days 7, 14, 17 and 24 as summarized in Table 3. On Day 7, 9/12 test areas that received an application of 0.1% or 0.5% ruboxistaurin gel were lighter than placebo (p-values 0.07); and 7/12 test areas that received an application of 1.0% ruboxistaurin gel were lighter than placebo. On Day 14, 9/12 test areas that received an application of 0.1% ruboxistaurin gel were lighter than placebo (p-value 0.07); and 7/12 and 8/12 test areas that received an application of 0.5% or 1.0% ruboxistaurin gel respectively were lighter than placebo. On Day 17, 9/12 test areas that received an application of 0.1%, 0.5% or 1.0% ruboxistaurin gel were lighter than placebo (p-values 0.07). On Day 24, 6/12 test areas that received an application of 0.1% ruboxistaurin gel, 9/12 test areas that received an application of 0.5% ruboxistaurin gel and 7/12 test areas that received an application of 1.0% ruboxistaurin gel were lighter than placebo.

TABLE 3
Proportion of the 12 test subjects in whom ruboxistaurin-
treated sites were lighter than placebo-treated sites
according to ITA scores on Days 7, 14, 17, and 24

| Test Article | Day 7 | Day 14 | Day 17 | Day 24 |
|---|---|---|---|---|
| 0.1 | 9/12* | 9/12* | 9/12* | 6/12 |
| | (p 0.07) | (p 0.07) | (p 0.07) | |
| 0.5 | 9/12* | 7/12 | 9/12* | 9/12* |
| | (p 0.07) | | (p 0.07) | (p 0.07) |
| 1 | 7/12 | 8/12 | 9/12* | 7/12 |
| | | | (p 0.07) | |
| Total | 25/37* | 24/36* | 27/36* | 22/36 |
| | (p 0.02) | (p 0.03) | (p 0.002) | |

Note:
Asterisks followed by a p value indicates the probability of this happening, e.g., by chance alone, if ruboxistaurin, e.g., had no effect. For example, p < 0.02 (as measured by a paired t test) means statistically there is a 2% chance that the indicated portion of the treated sites would have lighter readings if there were no effect of ruboxistaurin.

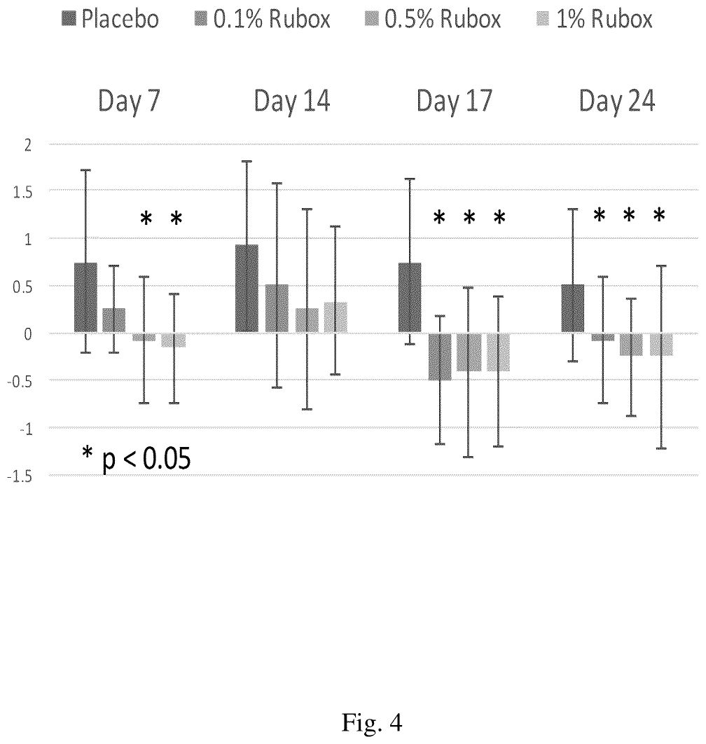

As depicted in FIG. 4, IDGA assessment of all test areas on subjects at Days 7, 14, 17, and 24 revealed statistically significant (p<0.05) lesser pigmentation, e.g., lighter skin, in test areas in which ruboxistaurin gel was applied vs. placebo. A lower IDGA value is indicative of lesser pigmentation (see Table 2 above). This decreased pigmentation was best appreciated on day 17 and 24 after UV irradiation when the erythema, e.g., sunburn, had faded completely and did not compete with the investigator's perception of brown (tan) color.

All of the doses of ruboxistaurin gel tested in this study were well tolerated under occlusion. No toxicity, application site reaction or irritation was observed in any of the subjects.

The results from this trial suggest that, in some embodiments, topical application of a ruboxistaurin gel formulation, e.g., at least 0.1% ruboxistaurin gel, can reduce skin darkening and pigmentation, e.g., melanogenesis, induced by, e.g., UV radiation. In some embodiments, topical application of a ruboxistaurin gel formulation, e.g., at least 0.1% ruboxistaurin gel, can result in a decrease in tyrosinase production of melanin.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating melasma in a human subject, comprising topically administering a composition comprising 0.05% to 1% by weight soluble ruboxistaurin mesylate to the human subject, thereby treating melasma in the human subject.

2. The method of claim 1, wherein treating melasma comprises decreasing healthy or non-disease state skin pigmentation.

3. (canceled)

4. The method of claim 1, wherein administering comprises contacting a device or a dermal patch comprising the composition with the human subject's skin.

5. (canceled)

6. (canceled)

7. (canceled)

8. The method of claim 1, wherein the administering is performed once or two times a day.

9. The method of claim 8, wherein the administering is performed for two months, three months, or four months.

10. The method of claim 1, wherein the administering comprises applying the ruboxistaurin mesylate to an area of the skin affected by the melasma.

11. The method of claim 1, wherein the treating reduces or inhibits melanogenesis in the subject.

12. The method of claim 11, wherein the treating reduces or inhibits melanogenesis by at least 10% compared to prior to administering the ruboxistaurin mesylate, as measured by a chromameter.

13. A topical gel composition comprising 0.05% to 1% by weight soluble ruboxistaurin mesylate.

14. (canceled)

15. (canceled)

16. (canceled)

17. (canceled)

18. (canceled)

19. (canceled)

20. A method of decreasing pigmentation of a hair of a human subject, comprising, topically applying an effective amount of 0.05% to 1% by weight ruboxistaurin mesylate to the hair or hair bearing skin of the human subject, thereby decreasing the pigmentation of the hair.

21. The method of claim 20, wherein at least a portion of the hair is artificially colored.

22. The method of claim 1, wherein the composition is formulated as a gel.

23. The method of claim 1, wherein the melasma is associated with ultra-violet (UV) radiation exposure.

24. The method of claim 1, wherein the administering is performed on the face of the human subject.

25. The topical gel composition of claim 13, comprising a solvent selected from ethyl alcohol, methylene chloride, isopropanol, acetone, ethylene glycol monoethyl ether, diethylene glycol monobutyl ether, diethylene glycol monoethyl ether, dimethyl sulphoxide, dimethyl formamide, tetrahydrofuran, propylene glycol, butylene glycol, dipropylene glycol, ethoxylated or propoxylated diglycol, cyclic polyol, or combinations thereof.
